(19) United States
(12) Patent Application Publication
(10) Pub. No.: US 2026/0265334 A1
(43) Pub. Date: Sep. 10, 2026

(54) STAPLED PEPTIDES

(71) Applicant: THE FLOREY INSTITUTE OF NEUROSCIENCE AND MENTAL HEALTH, Parkville (AU)

(72) Inventors: Akhter HOSSAIN, Parkville (AU); John FURNESS, Parkville (AU)

(21) Appl. No.: 19/164,776

(22) PCT Filed: Mar. 15, 2024
(86) PCT No.: PCT/AU2024/050230

§ 371 Date: Sep. 12, 2025

(30) Foreign Application Priority Data
Mar. 16, 2023 (AU) 2023900723

(51) Int. Cl. C07K 14/65; A61K 38/00; A61P 3/04

(52) U.S. Cl. CPC C07K 14/65; A61P 3/04; A61K 38/00

(57) ABSTRACT

The present disclosure relates to a peptide comprising two α,α-disubstituted amino acid residues, wherein: the peptide is comprises an α-helix; and the α,α-disubstituted amino acids each comprise as their α-substituents both of: a) an alkyl, cycloalkyl or alkenyl group; and b) an aromatic group capable of π-π stacking with the aromatic group of the other α,α-disubstituted amino acid residue, and wherein the two α,α-disubstituted amino acid residues are located at residue positions such that, in the α-helix, their aromatic groups may engage in π-π stacking with one another.

### CROSS-REFERENCE TO RELATED APPLICATIONS

The present application claims priority from Australian Provisional Patent Application No. 2023900723, the contents of which are incorporated herein by reference in their entirety.

### CROSS-REFERENCE TO SEQUENCE LISTING

The entire contents of the electronic submission of the sequence listing is incorporated by reference in its entirety for all purposes.

### TECHNICAL FIELD

The present disclosure broadly relates to stapled peptides comprising an alpha helical structure, and to uses of such peptides. Exemplary embodiments relate to peptides comprising two α-methyl-L-phenylalanine residues.

### BACKGROUND OF THE DISCLOSURE

Peptides and peptidomimetics are attractive alternatives to small molecule drugs because of their high target specificity and low toxicity profiles. Peptides are functional subunits of proteins and the α-helix is the most abundant peptide structure at the protein interaction interfaces that control cellular functions. When these π-helical peptides are excised from proteins they typically lose their bioactive structure due to the loss of peripheral interactions within the parent molecule and as a result, the unstructured peptides are unable to target protein interfaces. Recently developed peptide chemistry techniques attempt to develop peptidomimetics by reconstructing peptides into their natural π-helical conformation by chemical means (e.g., stapling).

A number of stapling strategies including lactam, triazole, thioether, and thioacetal bridges have been described to stabilize π-helical peptides (Moiola, M. et al., Stapled peptides-A useful improvement for peptide-based drugs. Molecules 2019, 24; and Andrews, M. J. I. et al., Forming stable helical peptides using natural and artificial amino acids. Tetrahedron 1999, 55, 11711-11743). However, such stapling strategies face problems such as low yield, poor solubility and unknown geometry (E/Z). One particular stapling method which has been recently studied is a hydrocarbon (HC)-stapling strategy which generates peptides following ruthenium-catalyzed (Grubb's catalyst) ring-closing metathesis (RCM) reactions (Walensky, L. D. et al., Hydrocarbon-stapled peptides: principles, practice, and progress. J Med Chem 2014, 57, 6275; Schafmeister, C. E. et al., An all-hydrocarbon cross-linking system for enhancing the helicity and metabolic stability of peptides. J Am Chem Soc 2000, 122, 5891-5892). The yield of the RCM product can be low if the side-chain orientation of the alkene pair is not in close proximity, and the separation of Grubb's catalyst from the product by high performance liquid chromatography (HPLC) is difficult. It is also challenging to control Cis-Trans (E/Z) isomerism and distinguish between the isomers by solution NMR spectroscopy, and the peptides are known to have poor oral bioavailability. A harsh hydrogenation reaction followed by RCM can produce one saturated product but with a low overall yield. Other α-helix-stabilizing methods (e.g., lactam, triazole, thioacetal, etc.) can also be low yielding as they require two-step synthesis (e.g. SPPS and then cyclization) and purification. While covalent stapling methods improve the helicity and stability of peptides, the rigid nature of covalent stapling may also cause disruption of the network of stabilizing intramolecular interactions present in the bound state of the native peptides and thus peptides sometimes (depending on the target and peptide itself) may not bind with the target with high affinity (Okamoto, T. et al. ACS Chemical Biology 2013, 8, 297-302. PMID 23151250). Therefore, a novel, high-yielding non-covalent stapling strategy with one-step synthesis and purification is required to provide peptides that are less rigid and are amenable to adopting an induced structure more optimal for binding with a target, in order to improve drug-target interactions and accelerate peptide-based drug development.

### SUMMARY OF THE DISCLOSURE

According to a first aspect, the present disclosure provides a peptide comprising two α,α-disubstituted amino acid residues, wherein:
the peptide comprises an α-helix; and
the α,α-disubstituted amino acids each comprise as their α-substituents both of:
an alkyl, cycloalkyl or alkenyl group; and;
an aromatic group capable of π-π stacking with the aromatic group of the other α,α-disubstituted amino acid residue,
and wherein the two α,α-disubstituted amino acid residues are located at residue positions such that, in the α-helix, their aromatic groups may engage in π-π stacking with one another.

In some embodiments, the two α,α-disubstituted amino acid residues are located at residue positions i and i+x, wherein x is 3, 4, 6, 7, 8, 9, 10, 11 or 12. In some particular embodiments, the two α,α-disubstituted amino acid residues are located at residue positions i and i+x, wherein x is 3, 4, 6, 7, or 8. In some particular embodiments, the two α,α-disubstituted amino acid residues are located at residue positions i and i+x, wherein x is 3 or 4.

In some embodiments, the peptide is a single-chain peptide.

In some embodiments, the alkyl, cycloalkyl or alkenyl group is a C₁-C₈ alkyl, cycloalkyl or alkenyl group, a C₁-C₆ alkyl, cycloalkyl or alkenyl group or a C₁-C₄ alkyl, cycloalkyl or alkenyl group. In some particular embodiments, the alkyl group is methyl.

In some embodiments, the aromatic group is a benzyl group.

In some particular embodiments, at least one of the α,α-disubstituted amino acid residues is α-methyl-L-phenylalanine. In some embodiments, two of the α,α-disubstituted amino acid residues are α-methyl-L-phenylalanine.

In some embodiments, the peptide is selective for a G protein-coupled receptor, preferably wherein the G protein-coupled receptor is an RXFP receptor.

In some embodiments, the peptide is selective for the RXFP3 receptor or the RXFP4 receptor.

In some embodiments, the peptide is an analogue of the B chain of an insulin-like peptide or a fragment thereof.

For example, in some embodiments, the peptide is an analogue of the B chain of H3 relaxin/insulin-like peptide 7 or a fragment thereof. In some embodiments, the peptide is an agonist of the RXFP3 receptor. In some such embodiments, the peptide is a peptide according to SEQ ID NO. 4, or a variant thereof comprising at least about 80% sequence identity to SEQ ID NO. 4. In some embodiments, the peptide is an antagonist of the RXFP3 receptor. In other particular embodiments, the peptide is a peptide according to SEQ ID NO. 22, or a variant thereof comprising at least about 80% sequence identity to SEQ ID NO. 4.

In some alternative embodiments, the peptide is an analogue of the B chain of insulin-like peptide 5 or a fragment thereof. In some embodiments, the peptide is an agonist of the RXFP4 receptor. In some embodiments, residues 20 and 21 are each substituted with Gly. In some particular embodiments, the peptide is a peptide according to SEQ ID NO. 16, or a variant thereof comprising at least about 80% sequence identity to SEQ ID NO 16.

In some embodiments, residue 19 is substituted with glutamic acid. In some particular embodiments, the peptide is a peptide according to SEQ ID NO. 26, or a variant thereof comprising at least about 80% sequence identity to SEQ ID NO 26.

According to a second aspect, the present disclosure relates to a pharmaceutical composition comprising a peptide according to the first aspect and a pharmaceutically acceptable carrier, excipient, diluent, vehicle, and/or adjuvant.

According to a third aspect, the present disclosure relates to a method of treating or preventing a condition associated with RXFP activity, preferably RXFP3 or RXFP4 activity, comprising administering to a subject in need thereof an effective amount of a peptide according to the first aspect or a pharmaceutical composition according to the second aspect.

In one such embodiment, the condition is associated with RXFP3 activity, and preferably the peptide is an analogue of the B chain of insulin-like peptide 7 or a fragment thereof. In a particular embodiment, the condition associated with RXFP3 activity is selected from an eating disorder, weight loss, weight gain, or obesity. In another particular embodiment, the condition associated with RXFP3 activity is pain. In another particular embodiment, the condition associated with RXFP3 activity is a neuropsychiatric disorder. In one example, the neuropsychiatric disorder is selected from anxiety or depression.

In another such embodiment, the condition is associated with RXFP4 activity, and preferably the peptide is an analogue of the B chain of insulin-like peptide 5 or a fragment thereof. In a particular embodiment, the condition associated with RXFP4 is a colon motility disorder.

According to a fourth aspect, the present disclosure relates to use of a peptide according to the first aspect or a pharmaceutical composition according to the second aspect, in the manufacture of a medicament for the treatment or prevention of a condition associated with RXFP activity.

In one such embodiment, the condition is associated with RXFP3 activity, and preferably the peptide is an analogue of the B chain of insulin-like peptide 7 or a fragment thereof. In a particular embodiment, the condition associated with RXFP3 activity is selected from an eating disorder, weight loss, weight gain, or obesity. In another particular embodiment, the condition associated with RXFP3 activity is pain. In another particular embodiment, the condition associated with RXFP3 activity is a neuropsychiatric disorder. In one example, the neuropsychiatric disorder is selected from anxiety or depression.

In another such embodiment, the condition is associated with RXFP4 activity, and preferably the peptide is an analogue of the B chain of insulin-like peptide 5 or a fragment thereof. In a particular embodiment, the condition associated with RXFP4 is a colon motility disorder.

### BRIEF DESCRIPTION OF THE FIGURES

Exemplary embodiments of the present disclosure are described herein, by way of non-limiting example only, with reference to the following drawings.

FIG. 1. Primary structures of human relaxin 3 (H3 relaxin) (A) (SEQ ID NOs 1 and 2) and its B-chain analogues used in Example 1: hydrocarbon (HC)-stapled Peptide 5 (B10-27(13/17HC)) (B) (SEQ ID NO. 3); Novel designed peptides B10-27(13/17αF) (C) (SEQ ID NO. 4); B10-27(13/17F) (D) (SEQ ID NO. 5) and B10-27 (E) (SEQ ID NO. 6).

FIG. 2. Helical wheel, viewing the helical region of the H3 relaxin B-chain from N to C-terminus.

FIG. 3. Characterization of helical structure of the target peptide (A) and (B) 2D ¹H ¹H NOESY spectra of the target (H3B10-27 (13/17αF) (SEQ ID NO. 4) and the control peptide (H3B10-27(13/17F) (SEQ ID NO. 5), respectively. Boxed in (A) are assigned short-range NOEs that are observed in the target peptide but absent in the control. (C) Summary of sequential and short-range NOES assigned for the target peptide. (D) The 20 low-energy structures calculated for the target peptide: highlighting the orientation and proximity of the phenyl rings of αF 13 and 17 in the target peptide. (E) Superposition of the NMR structure of H3 relaxin (SEQ ID NOs 1 and 2) and the target peptide, overlaying key residues such as R12, Ile15, R16, I19 and F20 essential for RXFP3 binding. (F) Superposition of the NMR structure of H3 relaxin, the B10-27(13/17 αF) peptide and Peptide 5 (SEQ ID NO. 3) overlaying key residues such as R12, 115, R16, I19 and F20 essential for binding to RXFP3; αF 13 and 17 residues and HC stapling in Peptide 5 are highlighted, responsible for their respective helical structures.

FIG. 4. Serum stability assay of H3B10-27 (13/17αF) (SEQ ID NO. 4) and H3B10-27 (SEQ ID NO. 6) at 37° C.

FIG. 5. Binding and agonist activity of peptides in CHO-K1-RXFP3 cells. (A) Competition binding curves of increasing concentrations of peptides in competition with 5 nM Eu-B1-22R; (B) Dose-response curves demonstrating inhibition of forskolin-induced cAMP activity.

FIG. 6. Binding and agonist activity of peptides in CHO-K1-RXFP4 cells. (A) Competition binding curves of increasing concentrations of peptides in competition with 5 nM Eu-R3/15; (B) Dose-response curves demonstrating inhibition of forskolin induced cAMP activity.

FIG. 7. RXFP3-NLuc construct signalled in response to an RXFP3 agonist, R3/15, with identical potency and efficacy compared to untagged RXFP3.

FIG. 8. Ligand induced β-arrestin2 recruitment. Area under the curve (AUC) dose-response curves of ligand-induced (A) RXFP3/0-arrestin recruitment NanoBRET responses, (B) RXFP4/0-arrestin recruitment NanoBRET responses.

FIG. 9. Ligand-induced β-arrestin recruitment measured by NanoBRET between nanoluciferase-tagged RXFP3 (RXFP3-NL) and Venus-tagged-o-arrestin2 (0-Arr2-Venus). Real-time NanoBRET measurement of the dose-dependent (A) R3/I5 (B) Peptide 5 (SEQ ID NO. 3) and (C) H3B10-27(13/17αF) (SEQ ID NO. 4) induced interaction of RXFP3-NL and β-Arr2-Venus.

FIG. 10. Ligand-induced β-arrestin recruitment measured by NanoBRET between nanoluciferase-tagged RXFP4 (RXFP4-NL) and Venus-tagged-β-arrestin2 (0-Arr2-Venus). Real-time NanoBRET measurement of the dose-dependent (A) R3/I5 (B) Peptide 5 (SEQ ID NO. 3) and (C) H3B10-27(13/17αF) (SEQ ID NO. 4) induced interaction of RXFP4-NL and β-Arr2-Venus.

FIG. 11. Effect of H3B10-27(13/17αF) (SEQ ID NO. 4) on respiratory rate and the arterial chemoreceptor reflex in an in situ perfused rat brainstem preparation. (A) Control recording of phrenic nerve activity (PNA) before and after evoking the arterial chemoreceptor reflex with a bolus injection of sodium cyanide (NaCN, 0.2 ml, 0.01%) in the perfusion circuit (black arrow, asterisks). (B). Effect of systemic application of 2 μM H3B10-27(13/17αF) on PNA burst rate (blue) and NaCN tachypnea (red). (C,D) Group data, n=6; *p≤0.01.

FIG. 12. Effect of icv infusion of different amounts (0.1-4 nmol in 5 μl) of H3B10-27(13/17αF) (SEQ ID NO. 4) on chow consumption in adult, male rats, within a 120 min period post-treatment. An icv infusion of A2 peptide (Shabanpoor, F. et al., Minimization of human relaxin-3 leading to high-affinity analogues with increased selectivity for relaxin-family peptide 3 receptor (RXFP3) over RXFP1. J Med Chem 2012, 55, 1671-1681) (1 nmol/5 μl) is illustrated for comparative purposes. Values are mean SEM. ns, non-significant; ^{#}p≤0.05, and ^{##}p≤0.01, Mann-Whitney test, *p≤0.05, t-test.

FIG. 13. NMR structure of human INSL5 (SEQ ID NOS 7 and 8).

FIG. 14. % Forskolin activity of INSL5 (SEQ ID NOs 7 and 8), INSL5-A13: B7-24 (reduced two chain analogue) (SEQ ID NOs 11 AND 12), INSL5-A13: B7-24_G20/21 (two-chain G20/21 analogue) (SEQ ID NOs 13 and 14) and the linear B chain (SEQ ID NO. 15).

FIG. 15. Schematic diagram of A) INSL5 structure (SEQ ID NOs 7 and 8) B) random coil structure of linear B chain (SEQ ID NO. 15) C) alpha helical structure of stapled B chain (SEQ ID NO. 16).

FIG. 16. Serum stability results of INSL5 analogues as studied in Example 7.

FIG. 17. A) Eu-R4/15 binding curves and B) cAMP inhibition assay for INSL5-A13 (two chain agonist) (SEQ ID NOs 9 and 10), linear B chain (SEQ ID NO. 15) and hydrocarbon stapled analogue (SEQ ID NO. 18).

FIG. 18. A) Eu-R3/15 binding and B) cAMP inhibition assay curves for linear B chain (SEQ ID NO. 15) and lactam bridge stapled analogue (SEQ ID NO. 19).

FIG. 19. A) Eu-R3/15 binding and B) cAMP inhibition assay binding curves for linear B chain (SEQ ID NO. 15) and thioacetal stapled analogue (SEQ ID NO. 20).

FIG. 20. A) Eu-R3/15 binding and B) cAMP inhibition assay binding curves for linear B chain (SEQ ID NO. 15) and disulfide stapled analogue (SEQ ID NO. 21).

FIG. 21. A) Eu-R3/15 binding and B) cAMP inhibition assay binding curves for linear B chain (SEQ ID NO. 19) and αF stapled analogue (SEQ ID NO. 16).

FIG. 22. Acceleration of colorectal propulsion as studied in Example 9.

FIG. 23. Paw withdrawal threshold in rat CFA model demonstrating reduced pain with RXFP3 agonist administration.

FIG. 24. Binding and agonist activity of peptides in CHO-K1-RXFP3 cells. (A) Competition binding curves of increasing concentrations of peptides in competition with 5 nM Eu-B1-22R; (B) Dose-response curves demonstrating inhibition of forskolin-induced cAMP activity.

FIG. 25. Binding and agonist activity of peptides in CHO-K1-RXFP4 cells. (A) Competition binding curves of increasing concentrations of peptides in competition with 5 nM Eu-R3/15; (B) Dose-response curves demonstrating inhibition of forskolin induced cAMP activity.

FIG. 26. Dose-response curves demonstrating antagonism of R3/15 function, (inhibition of forskolin induced cAMP activity).

FIG. 27. Effect of antagonist on agonist-induced chow consumption in adult rats.

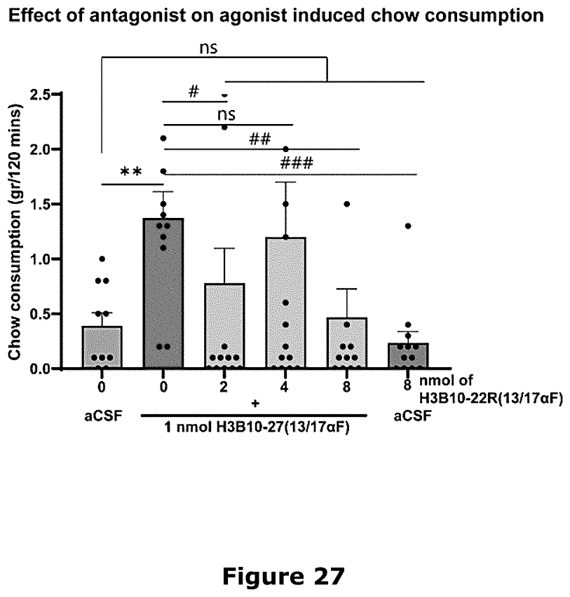

FIG. 28. Binding and agonist activity of peptides in CHO-K1-RXFP4 cells. (A) Competition binding curves of increasing concentrations of peptides in competition with 2 nM SmBiT R3/I5; (B) Dose-response curves demonstrating inhibition of forskolin induced cAMP activity.

### KEY TO SEQUENCE LISTING

SEQ ID NO 1: amino acid sequence of H3-relaxin A chain.

SEQ ID NO 2: amino acid sequence of H3-relaxin B chain.

SEQ ID NO 3: an 18 amino acid sequence of RXFP3 agonist B10-27 comprising a hydrocarbon staple (13/17HC).

SEQ ID NO 4: an 18 amino acid sequence of RXFP3 agonist B10-27 comprising two αF residues (13/17αF).

SEQ ID NO 5: an 18 amino acid sequence of RXFP3 agonist B10-27 comprising F substitution at residues 13 and 17 (13/17F).

SEQ ID NO 6: an 18 amino acid sequence of RXFP3 agonist native B10-27.

SEQ ID NO 7: a 21 amino acid sequence of INSL5 A chain.

SEQ ID NO 8: a 24 amino acid sequence of INSL5 B chain.

SEQ ID NO 9: a 14 amino acid sequence of the A chain of an INSL5 analogue.

SEQ ID NO 10: a 24 amino acid sequence of the B chain of an INSL5 analogue.

SEQ ID NO 11: a 14 amino acid sequence of the A chain of an INSL5 analogue.

SEQ ID NO 12: an 18 amino acid sequence of B chain of an INSL5 analogue.

SEQ ID NO 13: a 14 amino acid sequence of the A chain of an INSL5 analogue.

SEQ ID NO 14: an 18 amino acid sequence of B chain of an INSL5 analogue.

SEQ ID NO 15: an 18 amino acid sequence of INSL5 B chain analogue.

SEQ ID NO 16: an 18 amino acid sequence RXFP4 agonist comprising two aF residues.

SEQ ID NO 17: an acetylated 18 amino acid sequence RXFP4 agonist comprising two aF residues.

SEQ ID NO 18: an acetylated 18 amino acid sequence RXFP4 agonist comprising a hydrocarbon staple.

SEQ ID NO 19: an 18 amino acid sequence RXFP4 agonist comprising a lactam bridge staple.

SEQ ID NO 20: an acetylated 18 amino acid sequence RXFP4 agonist comprising a thioacetal staple.

SEQ ID NO 21: an acetylated 18 amino acid sequence of a disulfide bridge stapled peptide.

SEQ ID NO 22: a 14 amino acid sequence RXFP3 antagonist comprising two αF amino acids.

SEQ ID NO 23: a 23 amino acid sequence RXFP3 antagonist.

SEQ ID NO 24: a 14 amino acid sequence RXFP3 antagonist.

SEQ ID NO 25: a 14 amino acid sequence RXFP3 antagonist.

SEQ ID NO 26: an 18 amino acid sequence RXFP4 agonist comprising two aF residues.

### DETAILED DESCRIPTION

With regards to the definitions provided herein, unless stated otherwise, or implicit from context, the defined terms and phrases include the provided meanings. Unless explicitly stated otherwise, or apparent from context, the terms and phrases below do not exclude the meaning that the term or phrase has acquired by a person skilled in the relevant art. The definitions are provided to aid in describing particular embodiments, and are not intended to limit the claimed invention, because the scope of the invention is limited only by the claims. Furthermore, unless otherwise required by context, singular terms shall include pluralities and plural terms shall include the singular.

All publications discussed and/or referenced herein are incorporated herein in their entirety.

Any discussion of documents, acts, materials, devices, articles or the like which has been included in the present specification is solely for the purpose of providing a context for the present disclosure. It is not to be taken as an admission that any or all of these matters form part of the prior art base or were common general knowledge in the field relevant to the present disclosure as it existed before the priority date of each claim of this application.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout this specification, unless the context requires otherwise, the word “comprise”, or variations such as “comprises” or“comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

In the context of this specification, the term “about” is understood to refer to a range of numbers that a person of skill in the art would consider equivalent to the recited value in the context of achieving the same function or result.

In the context of this specification, the terms “a” and “an” refer to one or to more than one (i.e. to at least one) of the grammatical object of the article. By way of example, “an element” means one element or more than one element.

As used herein the terms “treating”, “treatment”, “treating”, “reduce”, “reducing”, “prevent” “preventing” and “prevention” and the like refer to any and all applications which remedy, or otherwise hinder, retard, or reverse the progression of, an infection, condition or disease or at least one symptom of an infection, condition or disease, including reducing the severity of an infection, condition or disease. Thus, the terms “treat”, “treating”, “treatment”, do not necessarily imply that a subject is treated until complete elimination of the infection or recovery from a disease or condition. For the avoidance of doubt, it will be understood that the terms “treat”, “treating” and “treatment” also encompass “stabilising” or “stabilisation” and “managing” or “management” of a disease or condition. Similarly, the terms “prevent”, “preventing”, “prevention” and the like refer to any and all applications that prevent the establishment or onset of an infection, condition or disease or otherwise delay the onset of an infection, condition or disease.

The term “optionally” is used herein to mean that the subsequently described feature may or may not be present or that the subsequently described event or circumstance may or may not occur. Hence the specification will be understood to include and encompass embodiments in which the feature is present and embodiments in which the feature is not present, and embodiments in which the event or circumstance occurs as well as embodiments in which it does not.

As used herein the terms “effective amount” and “effective dose” include within their meaning a non-toxic but sufficient amount or dose of a peptide to provide the desired effect. The exact amount or dose required will vary from subject to subject depending on factors such as the species being treated, the age and general condition of the subject, the severity of the condition being treated, the particular peptide being administered and the mode of administration and so forth. Thus, it is not possible to specify an exact “effective amount” or “effective dose”. However, for any given case, an appropriate “effective amount” or “effective dose” may be determined by one of ordinary skill in the art using only routine experimentation.

The term “subject” as used herein refers to mammals and includes humans, primates, livestock animals (e.g. sheep, pigs, cattle, horses, donkeys), laboratory test animals (e.g. mice, rabbits, rats, guinea pigs), performance and show animals (e.g. horses, livestock, dogs, cats), companion animals (e.g. dogs, cats) and captive wild animals. In particular embodiments, the subject is a human.

The term “peptide” means a polymer made up of amino acids linked together by peptide bonds. The terms “polypeptide” or “protein” may also be used to refer to such a polymer although in some instances a polypeptide or protein may be longer (i.e. composed of more amino acid residues) than a peptide. Typically, the term peptide is used to define a sequence of amino acids of up to about 70 amino acids. The term “protein” may typically be used to define a sequence of amino acids of greater than about 70 amino acids. Nevertheless, as will be familiar to a skilled person in the art, the terms may be used interchangeably herein and readily understood from context of their use.

The term “selective” when used in the context of the ability of a peptide of the present disclosure to bind a particular receptor, for example the RXFP3 receptor, means that the peptide binds that receptor at significantly higher frequency than it binds other receptors, for example the RXFP4 receptor. In some embodiments, peptides of the present disclosure that are selective for a particular receptor, are also specific for that particular receptor. A peptide of the present disclosure that is “specific” for a particular receptor is one that possesses no discernible activity at any other receptor. Thus, a peptide of the present disclosure that is “specific” (e.g. RXFP3) is, by definition, selective (e.g. for RXFP3).

The term “G protein coupled receptor(s)” or “GPCR(s)” encompasses all members of the GPCR superfamily of receptors, which are well known to the skilled person. GPCR typically refers to a target receptor that, when expressed by a cell, associates with a G-protein (e.g. a protein that hydrolyses GTP). In some embodiments the GPCR is a “seven transmembrane segment receptor”, referring to a protein that structurally comprises seven hydrophobic transmembrane spanning regions.

The term “agonist” refers to a ligand (e.g. peptide) that enhances or increases the activity (e.g. signalling activity) of a target (e.g. receptor) to which they bind or associate, upon binding or association. Full agonists are capable of maximal receptor stimulation; partial agonists are unable to elicit full activity even at saturating concentrations. Partial agonists may in some instances be referred to as “blockers”, whereby they prevent the binding of more robust agonists.

The term “antagonist” refers to a ligand (e.g. peptide) that inhibits or reduces the activity (e.g. signalling activity) of a target (e.g. receptor) to which they bind or associate, upon binding or association. An “antagonist” may also be referred to as a “blocker” in some instances because of its ability to prevent binding of other ligands and, therefore, block agonist-induced activity.

As used herein “relaxin” relates to the family of peptide hormones within the insulin superfamily, and are known to the skilled person (see e.g. Patil, N. A. et al., Relaxin family peptides: structure-activity relationship studies, British Journal of Pharmacology, 174: 950-961). In humans, the relaxin family includes seven peptides with high structural similarity but low sequence similarity: relaxin 1, 2, and 3 the insulin-like peptides INSL3, INSL4, INSL5, and INSL6. In humans, relaxin 1, 2 and 3 may be referred to as H1-relaxin, H2-relaxin or H3 relaxin respectively. H3 relaxin is also known as INSL7. H2 relaxin, INSL3, H3 relaxin and INSL5 signal through RXFP1, RXFP2, RXFP3 and RXFP4 receptors respectively. Peptides of the present disclosure may be an analogue of a relaxin. In some embodiments, the peptide of the present disclosure is an analogue of the B chain of a relaxin. In some embodiments, the relaxin is a human relaxin. In some embodiments, relaxin is an insulin-like peptide (INSL). In some examples, the relaxin is INSL7 (H3 relaxin), INSL5, INSL3, INSL4, INSL6, relaxin 1 or relaxin 2. In some examples, the relaxin is INSL7 (H3 relaxin) or INSL5. In some examples, the relaxin is INSL7 (H3 relaxin). In some examples, the relaxin is INSL5. In some examples, the relaxin is H2 relaxin. In some examples, the relaxin is INSL3.

As used herein “relaxin family peptide receptor”, or “RXFP”, relates to any member of the family of relaxin family peptide receptors, which are known to the skilled person as relaxin receptors, having the biological activity of mediating G-protein coupled signalling from relaxin peptide hormones (see e.g. Patil, N. A. et al., Relaxin family peptides: structure-activity relationship studies, British Journal of Pharmacology, 174: 950-961). In some embodiments the RXFP is human RXFP. In some embodiments, the RXFP receptor is RXFP1, RXFP2, RXFP3 or RXFP4. In some embodiments, the RXFP receptor is RXFP1. In some embodiments, the RXFP receptor is RXFP2. In some embodiments, the RXFP receptor is RXFP3. In some embodiments, the RXFP receptor is RXFP4. In some embodiments, the RXFP receptor is RXFP3 or RXFP4.

The present disclosure relates to a peptide comprising two α,α-disubstituted amino acid residues, wherein:
the peptide comprises an α-helix; and
the α,α-disubstituted amino acids each comprise as their α-substituents both of:
a) an alkyl, cycloalkyl or alkenyl group; and
b) an aromatic group capable of π-π stacking with the aromatic group of the other α,α-disubstituted amino acid residue,
and wherein the two α,α-disubstituted amino acid residues are located at residue positions such that, in the α-helix, their aromatic groups may engage in π-π stacking with one another.

The present inventors have surprisingly found that the incorporation of two residues of a α,α-disubstituted amino acid, α-methyl-L-phenylalanine (αF), results in a π-helical structure of the otherwise unstructured linear H3 relaxin B-chain and insulin-like peptide 5 (INSL5) B-chain which can be prepared in high yield and which retain biological activity at their respective receptors. In their native form, both H3 relaxin and insulin-like peptide 5 comprise two (A and B) chains linked by disulphide bridges, wherein the B-chain has an alpha-helical structure. Without wishing to be bound by theory, it is thought that, in the single-chain analogues developed by the present inventors, in addition to the α-methyl group in αF that places steric constraints on the peptide backbone that favours helical dihedral angles, additional hydrophobic or π-π interactions between the two aromatic phenyl rings oriented towards each other on the same side of the peptide lead to a non-covalently stapled helical peptide.

By way of the stapling method identified by the present inventors, the B chain alone can adopt the alpha-helical structure found in the native peptides and retain biological activity, allowing use as a minimised mimetic of the native peptides. H3 relaxin mimetics have potential application for the treatment of neuropsychiatric disorders such as anxiety and depression (Smith, C. M et al. Front Pharmacol 2014, 5, 46. 24711793), and INSL5 mimetics have potential application in treating constipation and colon motility disorders.

Compared to the larger native peptides and their analogues, these smaller mimetics may provide various advantages such as cheaper and easier manufacturing, since only one peptide chain requires manufacture, higher yield, and/or the presence or increase of oral availability. A further particular advantage of the peptides disclosed herein e.g. over other forms of stapled peptides is that they may be synthesised via a one-step high-yielding method (i.e. they do not require a cyclisation step) providing a single product (unlike e.g. hydrocarbon stapling methods with provide two products). Moreover, the produced stapled peptides have good water solubility. Furthermore, without intending to be limited by theory, it is believed that α,α-disubstituted amino acid stapled peptides (e.g. αF stapled peptides) utilize the helix-inducing properties of both α,α-disubstituted amino acids (e.g. α-methyl and phenyl moieties in αF) to engage in π-π stacking with one another. The advantages of such non-covalent a helical stapled peptides is that they are less rigid compared with the current stabilization generated stapled peptides, and thereby allow for an induced structure optimal for many drug-target interactions.

As discussed above, an existing method of preparing minimised peptides, using hydrocarbon stapling to lock peptides into an π-helical structure, involves a chemical reaction known as ring-closing metathesis (RCM) which results in a mixture of unsaturated compounds that are often difficult to separate and characterise. A harsh hydrogenation reaction followed by RCM can produce one saturated product but with a low overall yield. These peptides are also known to have poor oral bioavailability. In contrast, as demonstrated by the Examples below, the peptides of the present disclosure can be produced in high yield by relatively simple synthesis methods, whilst exhibiting high serum stability and efficient binding and activity at target receptors. Without wishing to be bound by theory, it is also thought that the peptides of the present disclosure may provide a less “stiff” peptide than those incorporating a hydrocarbon staple, potentially broadening the possible applications of the minimised peptide beyond those available to a hydrocarbon-stapled or any other covalently stapled peptide. While covalent stapling methods improve the helicity and stability of peptides, the rigid nature of covalent stapling may also cause disruption of the network of stabilizing intramolecular interactions present in the bound state of the native peptides and thus peptides sometimes may not bind with the target with high affinity (Okamoto, T. et al. ACS Chemical Biology 2013, 8, 297-302. PMID 23151250). In contrast, it is thought that the non-covalent stapling method of the present disclosure which produces a non-rigid helical structure may work on a range of other biologically important peptides.

A peptide of the present disclosure may be any peptide for which an π-helical structure is desired. As used herein, the term “peptide” may be used interchangeably with “protein” except where otherwise dictated by context. Suitable proteins or peptides will be familiar to a person skilled in the art. In some embodiments, the peptide may be an analogue, variant or derivative of a naturally occurring peptide or protein, including an analogue, variant or derivative of a subunit, for example a strand, of a naturally occurring peptide or protein, or of a functional fragment of naturally occurring peptide or protein, for example of a portion of the B-chain of an insulin-like peptide.

The peptides of the present disclosure may be capable of selectively or specifically binding and activating cell membrane receptors. Accordingly, in some embodiments, the peptide is selective or specific for a cell membrane receptor. In some embodiments, the peptide is selective for a cell membrane receptor. In some embodiments, the cell membrane receptor is a G-protein-coupled receptor. In some embodiments, the cell membrane receptor is a G-protein-coupled receptor, preferably an RXFP receptor.

In some embodiments, the peptide is selective for an RXFP receptor. In some embodiments, the peptide is selective for an RXFP receptor. In some embodiments, the peptide is selective for the RXFP1 receptor, RXFP2 receptor, RXFP3 receptor or RXFP4 receptor. In some embodiments, the peptide is selective for the RXFP3 receptor or RXFP4 receptor. In some embodiments, the peptide is selective for the RXFP1 receptor. In some embodiments, the peptide is selective for the RXFP2 receptor. In some embodiments, the peptide is selective for the RXFP3 receptor. In some embodiments, the peptide is selective for the RXFP4 receptor.

In some embodiments, the peptide is an agonist. Accordingly, in some embodiments, the peptide is an agonist of the RXFP1 receptor, RXFP2 receptor, RXFP3 receptor or RXFP4 receptor. In some embodiments, the peptide is an agonist of the RXFP3 receptor or RXFP4 receptor. In some embodiments, the peptide is an agonist of the RXFP1 receptor. In some embodiments, the peptide is an agonist of the RXFP2 receptor. In some embodiments, the peptide is an agonist of the RXFP3 receptor. In some embodiments, the peptide is an agonist of the RXFP4 receptor.

In some embodiments, the peptide is an antagonist. Accordingly, in some embodiments, the peptide is an antagonist of the RXFP1 receptor, RXFP2 receptor, RXFP3 receptor or RXFP4 receptor. In some embodiments, the peptide is an antagonist of the RXFP3 receptor or RXFP4 receptor. In some embodiments, the peptide is an antagonist of the RXFP1 receptor. In some embodiments, the peptide is an antagonist of the RXFP2 receptor. In some embodiments, the peptide is an antagonist of the RXFP3 receptor. In some embodiments, the peptide is an antagonist of the RXFP4 receptor.

Further exemplary proteins and peptides include, but are not limited to: BID BH3, BAD BH3, BIM BH3, MCL-1 BH3, PUMA BH3, p53, mastermind, BCL9, axin, p110α, borealin, EZH2, eIF4G, HIV-1 capsid, HIV-1 integrase, GP41 HR2 domain, lasioglossin III, melectin, CD81, esculentin-2EM, apolipoprotein A1, phospho-BAD BH3, nuclear receptor coactivator peptide 2, conantokins, galanin, neuropeptide Y, and nociception. Further examples of those peptides which may be used to prepare alpha helical peptides in accordance with the present disclosure include those disclosed in Harrison, R. S. et al. Proc Natl Acad Sci USA 2010, 107, 11686-11691. PMID 20543141 (Ac-(1,5-cyclo)-[KAAAD]-NH2, Ac-(1,5- and 6,10-cyclo)-[KAAAD]2-NH2 and Ac-(1,5-cyclo and 8,12-cyclo)-[KAAAD]AA-[KAAAD]-NH2), wherein K and D residues are replaced with αF residues for creating a one-turn and two-turn helix peptides. Variants, analogues, derivatives and functional fragments of these proteins and peptides may also be used.

In some embodiments, the peptide is an analogue of the B-chain of a relaxin, such as INSL7 (H3 relaxin), INSL5, INSL3, INSL4, INSL6, relaxin 2 (H2 relaxin) or relaxin 1.

In some embodiments, the peptide may be an analogue of the B-chain of an insulin-like peptide (INSL), such as INSL7 (H3 relaxin), INSL5, INSL3, INSL4, INSL6 or relaxin 2 (H2 relaxin).

In some embodiments, the insulin-like peptide is a human insulin-like peptide. In some embodiments, the peptide is an analogue of the B-chain of INSL7, INSL5, INSL3, or relaxin 2. In some embodiments, the peptide is an analogue of the B-chain of INSL7 or INSL5. In some embodiments, the peptide is an analogue of the B-chain of INSL7. In some embodiments, the peptide is an analogue of the B-chain of INSL5. In some embodiments, the peptide may be an analogue of the B-chain of INSL3. In some embodiments, the peptide may be an analogue of the B-chain of relaxin 2. It will be understood that such analogues, including may comprise further amino acid substitutions, insertions or deletions relative to the B-chain of the insulin-like peptide or relaxin from which they are derived, according to the present disclosure.

In some embodiments where the peptide is selective for the RXFP1 receptor, the peptide is an analogue of the B-chain of H2-relaxin. In some embodiments where the peptide is selective for the RXFP2 receptor, the peptide is an analogue of the B-chain of INSL3. In some embodiments where the peptide is selective for the RXFP3 receptor, the peptide is an analogue of the B-chain of INSL7. In some embodiments where the peptide is selective for the RXFP4 receptor, the peptide is an analogue of the B-chain of INSL5.

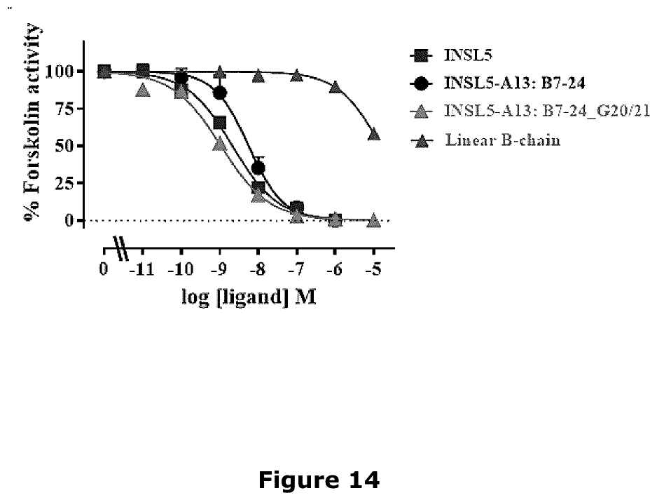

The present inventors have identified that substitution of each of the residues 20 and 21 in the B chain of INSL5 (Alanine and Serine in native INSL5) with Glycine gives a significant improvement in the binding and affinity to receptor RXFP4 (FIG. 14, Example 6). Accordingly, in some embodiments, the peptide is an analogue of the B-chain of INSL5 wherein residues 20 and 21 are substituted with Glycine.

In some embodiments, the peptide is an analogue of the B-chain of INSL5, wherein residue 19 is substituted with glutamic acid. In some embodiments, the peptide is an analogue of the B-chain of INSL5, wherein residues 20 and 21 are substituted with Glycine, and residue 19 is substituted with glutamic acid. In particular embodiments, the peptide may comprise the amino acid sequence set forth in SEQ ID NO:4, or a variant thereof. In particular embodiments, the peptide may comprise the amino acid sequence set forth in SEQ ID NO:16, or a variant thereof. In particular embodiments, the peptide may comprise the amino acid sequence set forth in SEQ ID NO:22, or a variant thereof. In particular embodiments, the peptide may comprise the amino acid sequence set forth in SEQ ID NO:4, SEQ ID NO:22, or a variant thereof.

In particular embodiments, the peptide may comprise the amino acid sequence set forth in SEQ ID NO:26, or a variant thereof. In particular embodiments, the peptide may comprise the amino acid sequence set forth in SEQ ID NO:16, SEQ ID NO:26 or a variant thereof. In particularly preferred embodiments, the peptide is a single chain-peptide. As discussed above and in the Examples below, an advantage of embodiments of the present disclosure is the ability to produce minimised mimetics of larger proteins or peptides, wherein the minimised peptides consist of a single stranded peptide yet retain the π-helical structure found in the native peptide even without supporting bonds and bridges being made to a secondary strand. Nonetheless, it will be appreciated that some embodiments of the disclosure may find use in a peptide or protein which comprise two or more strands. For example, the stapling technique of the present disclosure may be applied in one portion of a peptide, whilst, in another portion of the peptide strand, bonding or bridging with a further peptide strand is present.

In the present disclosure, reference to a named peptide or protein may be understood as including its derivatives, variants and/or functional fragments, unless specified otherwise or suggested otherwise by context.

As used herein, the term “derivative” is intended to encompass chemical modification to a peptide or protein or more amino acid residues of a peptide or protein, including chemical modification in vitro, for example by introducing a group in a side chain in one or more positions of a peptide, such as a nitro group in a tyrosine residue or iodine in a tyrosine residue, by conversion of a free carboxylic group to an ester group or to an amide group, by converting an amino group to an amide by acylation, by acylating a hydroxy group rendering an ester, by alkylation of a primary amine rendering a secondary amine, or linkage of a hydrophilic moiety to an amino acid side chain. Other derivatives may be obtained by oxidation or reduction of the side-chains of the amino acid residues in the peptide. Modification of an amino acid may also include derivation of an amino acid by the addition and/or removal of chemical groups to/from the amino acid, and may include substitution of an amino acid with an amino acid analog (such as a phosphorylated amino acid) or a non-naturally occurring amino acid such as a N-alkylated amino acid (e.g. N-methyl amino acid), D-amino acid, β-amino acid or γ-amino acid.

A variant or analogue of a peptide, such as a naturally occurring peptide or protein, in the context of the present specification, is a peptide or protein wherein one or more amino acids of the peptide sequence have been substituted for a different amino acid. In some embodiments, the protein or peptide of the present disclosure is a conservative variant of a naturally occurring or native peptide or protein, meaning the peptide or protein comprises one or more conservative amino acid substitutions. A “conservative amino acid substitution” is one in which an amino acid residue is replaced with another residue having a chemically similar or derivatised side chain. Families of amino acid residues having similar side chains, for example, have been defined in the art. These families include, for example, amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, the substitution of the neutral amino acid serine (S) for the similarly neutral amino acid threonine (T) would be a conservative amino acid substitution. Those skilled in the art will be able to determine suitable conservative amino acid substitutions that do not eliminate the functional properties of the peptide sequence required in the context of the present disclosure. In particular embodiments, the variant will possess at least about 80% identity to the sequence of which it is a variant. The sequence may be about 80%, 81%, 82%, 83%, 84%, 85%, 86%, 87%, 88%, 89%, 90%, 91%, 92%, 93%, 94%, 95%, 96%, 97%, 98% or 99% identical to the sequence of which it is a variant. In some embodiments, the variant comprises at least one amino acid substitution, at least one amino acid deletion, at least one amino acid insertion. In some embodiments, the variant comprises one, two, three, four or five amino acid substitutions. In some embodiments, the variant comprises one, two, three or four amino acid substitutions. In some embodiments, the variant comprises one, two, three amino acid substitutions. In some embodiments, the variant comprises one or two amino acid substitutions. In some embodiments, the variant comprises a single amino acid substitution. In some embodiments, the variant comprises one, two, three, four or five amino acid insertions. In some embodiments, the variant comprises one, two, three or four amino acid insertions. In some embodiments, the variant comprises one, two, three amino acid insertions. In some embodiments, the variant comprises one or two amino acid insertions. In some embodiments, the variant comprises a single amino acid insertion. In some embodiments, the variant comprises one, two, three, four or five amino acid deletions. In some embodiments, the variant comprises one, two, three or four amino acid deletions. In some embodiments, the variant comprises one, two, three amino acid deletions. In some embodiments, the variant comprises one or two amino acid deletions. In some embodiments, the variant comprises a single amino acid deletion. Numerous means are available, and will be known, to those skilled in the art for determining sequence identity, for example computer programs that employ algorithms such as BLAST (Basic Local Alignment Search Tool, Altschul et al., 1993, J. Mol. Biol. 215:403-410).

In some embodiments, a variant or an analogue of a naturally occurring peptide or protein (for example, an analogue of insulin-like peptide 5 or insulin-like peptide 7) will comprise two amino substitutions, such that the variant or analogue comprises two α,α-disubstituted amino acid residues. It will be understood that such variants or analogues of a naturally occurring peptide or protein that comprise two α,α-disubstituted amino acid residues, may further comprise one or more additional substitutions, insertions or deletions as described above.

Persons skilled in the art will be able to determine substitutions, deletions or insertions that retain the functional properties of the peptide sequence in the context of the present disclosure, and so determine a functional variant. Numerous means for determining such variants are known to persons skilled in the art, for example protein language models, such as ESM1b (Brandes, N. et al. Genome-wide prediction of disease variant effects with a deep protein language model, Nature Genetics (2023) 55, 1515-1522). It will be understood that a functional variant will enhance, retain or substantially retain the functional properties of the peptide sequence of which they are a variant of in the context of this disclosure. In some embodiments, the variant comprises an α-helix. In some embodiments, the aromatic groups of the two α,α-disubstituted amino acid residues of the variant may engage in π-π stacking with one another. In some embodiments, the variant is agonist of an RXFP receptor (e.g. RXFP1, RXFP2, RXFP3 or RXFP4). In some embodiments, the variant is antagonist of an RXFP receptor (e.g. RXFP1, RXFP2, RXFP3 or RXFP4. In some embodiments, the variant is specific for an RXFP receptor (e.g. RXFP1, RXFP2, RXFP3 or RXFP4). Methods for testing and/or confirming the specificity as well as ability of a variant to agonise and/or antagonise the function of a receptor are known in the art, and include by way of example and without limitation, such methods that are described herein, for example, those described in the Examples.

It will be understood that certain substitutions may provide a variant that is an agonist of the RXFP4 receptor. In some embodiments, the peptide is an analogue of the B-chain of INSL5 (e.g. a peptide according to SEQ ID NO. 16 or SEQ ID NO. 26), and further comprises a single amino acid substitution at a residue position selected from the group consisting of 7, 8, 9, 10, 12, 14, 19, 22, 23 and 24. In some such embodiments, the peptide is an analogue of the B-chain of INSL5, and further comprises 1, 2, 3, 4, 5, 6, 7, 8, 9 or 10 amino acid substitution(s) at a residue position selected from the group consisting of 7, 8, 9, 10, 12, 14, 19, 22, 23 and 24. In one embodiment, the peptide further comprises a single additional amino substitution. In one embodiment, the peptide further comprises one or two additional amino substitutions. In one embodiment, the peptide further comprises one, two, or three additional amino substitutions. In one embodiment, the peptide further comprises one, two, three or four additional amino substitutions. In some embodiments, the peptide is an analogue of the B-chain of INSL5, and further comprises a single amino acid substitution at a residue 7. In some embodiments, the peptide is an analogue of the B-chain of INSL5, and further comprises a single amino acid substitution at a residue 8. In some embodiments, the peptide is an analogue of the B-chain of INSL5, and further comprises a single amino acid substitution at a residue 9. In some embodiments, the peptide is an analogue of the B-chain of INSL5, and further comprises a single amino acid substitution at a residue 10. In some embodiments, the peptide is an analogue of the B-chain of INSL5, and further comprises a single amino acid substitution at a residue 12. In some embodiments, the peptide is an analogue of the B-chain of INSL5, and further comprises a single amino acid substitution at a residue 14. In some embodiments, the peptide is an analogue of the B-chain of INSL5, and further comprises a single amino acid substitution at a residue 19. In some embodiments, the peptide is an analogue of the B-chain of INSL5, and further comprises a single amino acid substitution at a residue 22. In some embodiments, the peptide is an analogue of the B-chain of INSL5, and further comprises a single amino acid substitution at a residue 23. In some embodiments, the peptide is an analogue of the B-chain of INSL5, and further comprises a single amino acid substitution at a residue 24. There is no particular limitation as to amino acids that are suitable for substitution at such positions. In some embodiments, the amino acid substitution is a conservative amino acid substitution. In some embodiments, the amino acid substitution is a non-natural amino acid substitution. It will understood that such further substitution is in addition to the substitution required such that the peptide comprises two α,α-disubstituted amino acid residues.

It will be understood that certain substitutions may provide a variant that is an agonist or an antagonist of the RXFP3 receptor. In some embodiments, the peptide is an analogue of the B-chain of INSL7 (e.g. a peptide according to SEQ ID NO. 4 or SEQ ID NO. 22), and further comprises a single amino acid substitution at a residue position selected from the group consisting of 10, 11, 12, 13, 15, 17, 22, 25, 26 and 27. In some such embodiments, the peptide is an analogue of the B-chain of INSL7, and further comprises 1, 2, 3, 4, 5, 6, 7, 8, 9 or 10 amino acid substitution(s) at a residue position selected from the group consisting of 10, 11, 12, 13, 15, 17, 22, 25, 26 or 27. In one embodiment, the peptide further comprises a single additional amino substitution. In one embodiment, the peptide further comprises one or two additional amino substitutions. In one embodiment, the peptide further comprises one, two, or three additional amino substitutions. In one embodiment, the peptide further comprises one, two, three or four additional amino substitutions. In some embodiments, the peptide is an analogue of the B-chain of INSL7, and further comprises a single amino acid substitution at a residue 10. In some embodiments, the peptide is an analogue of the B-chain of INSL7, and further comprises a single amino acid substitution at a residue 11. In some embodiments, the peptide is an analogue of the B-chain of INSL7, and further comprises a single amino acid substitution at a residue 12. In some embodiments, the peptide is an analogue of the B-chain of INSL7, and further comprises a single amino acid substitution at a residue 13. In some embodiments, the peptide is an analogue of the B-chain of INSL7, and further comprises a single amino acid substitution at a residue 15. In some embodiments, the peptide is an analogue of the B-chain of INSL7, and further comprises a single amino acid substitution at a residue 17. In some embodiments, the peptide is an analogue of the B-chain of INSL7, and further comprises a single amino acid substitution at a residue 22. In some embodiments, the peptide is an analogue of the B-chain of INSL7, and further comprises a single amino acid substitution at a residue 25. In some embodiments, the peptide is an analogue of the B-chain of INSL7, and further comprises a single amino acid substitution at a residue 26. In some embodiments, the peptide is an analogue of the B-chain of INSL7, and further comprises a single amino acid substitution at a residue 27. There is no particular limitation as to amino acids that are suitable for substitution at such positions. In some embodiments, the amino acid substitution is a conservative amino acid substitution. In some embodiments, the amino acid substitution is a non-natural amino acid substitution. It will understood that such further substitution is in addition to the substitution required such that the peptide comprises two α,α-disubstituted amino acid residues.

It will be understood that certain substitutions may provide a variant that is an antagonist of the RXFP4 receptor. In some embodiments, the peptide is an analogue of the B-chain of INSL5, and further comprises a single amino acid substitution at a residue position selected from the group consisting of 13, 26 and 27. In some such embodiments, the peptide is an analogue of the B-chain of INSL5, and further comprises 1, 2 or 3 amino acid substitution at a residue position selected from the group consisting of 13, 26 and 27. In one embodiment, the peptide further comprises a single additional amino substitution. In one embodiment, the peptide further comprises one or two additional amino substitutions. In one embodiment, the peptide further comprises one, two, or three additional amino substitutions. In some embodiments, the peptide is an analogue of the B-chain of INSL5, and further comprises a single amino acid substitution at a residue 13. In some embodiments, the peptide is an analogue of the B-chain of INSL5, and further comprises a single amino acid substitution at a residue 26. In some embodiments, the peptide is an analogue of the B-chain of INSL5, and further comprises a single amino acid substitution at a residue 27. There is no particular limitation as to amino acids that are suitable for substitution at such positions. In some embodiments, the amino acid substitution is a conservative amino acid substitution. In some embodiments, the amino acid substitution is a non-natural amino acid substitution. It will understood that such further substitution is in addition to the substitution required such that the peptide comprises two α,α-disubstituted amino acid residues.

As will be apparent to and readily understood by a skilled person in the art of peptides and protein, the terms “analogue”, “variant” and “derivative” may be used somewhat interchangeably, such as when referring to a peptide sequence which has undergone amino acid substitutions, including for other amino acids with side-chain substitutions. The meaning of the terms and the peptides to which they refer will be readily understood from the context of the specification.

Embodiments of the present disclosure also relate to the modification of functional fragments of peptides and proteins. As used herein, the term “functional fragment” refers to a fragment of a peptide or protein is a subsequence of the peptide that performs a similar function and retains substantially the same activity as the peptide or protein sequence from which the fragment is derived. References to analogues or variants of a naturally occurring peptide or protein may include analogues or variants of functional fragments or subunits thereof.

The terms “naturally occurring” and “native” refer to peptides and proteins as encoded by and produced from the genome of an organism.

The peptide or protein according to the present disclosure may be produced using any method known in the art, including synthetically or by recombinant techniques such as expression of nucleic acid constructs encoding the peptide or protein. For example, a peptide may be synthesised using solid phase peptide synthesis, for example the Fmoc-polyamide mode of solid-phase peptide synthesis, for example continuous flow Fmoc solid phase peptide synthesis. Other synthesis methods include solid phase t-Boc synthesis and liquid phase synthesis. Purification can be performed by any one, or a combination of, techniques such as re-crystallisation, size exclusion chromatography, ion-exchange chromatography, hydrophobic interaction chromatography and reverse-phase high performance liquid chromatography using, for example, acetonitrile/water gradient separation.

The peptides for use in the present disclosure may also be modified by natural or unnatural amino acids to improve their potency, selectivity, and stability. For example, the pharmacokinetic and/or pharmacodynamics properties (including in vivo half-life) can be improved by conjugating the peptides with fatty acids or larger proteins or polymers.

The peptide of the present disclosure comprises an π-helical structure. As described herein, this π-helical structure is the result of π-π stacking occurring between the two α,α-disubstituted amino acid residues forming a “staple” between residues of the peptide, combined with steric contributions from the α,α-disubstituted amino acid residues supporting α-helix formation. The entire peptide may be in an π-helical structure or, in some embodiments, only part of the peptide may be in an π-helical structure. The conditions under which the peptide comprises its π-helical structure include at least physiological conditions.

Peptides of the present disclosure comprise two α,α-disubstituted amino acid residues. The peptides of the present disclosure comprise at least two such residues, which interact by way of π-π stacking to form a ‘staple’ and provide an π-helical peptide structure. However, it will be appreciated that peptides of the present disclosure may comprise more than two such resides. For example, a peptide may comprise three or more such residues, each interacting by way of π-π stacking to their next nearest residues to form a ‘chain’ of π-π stacking along the peptide or a portion thereof. Alternatively, a peptide of the present disclosure may comprise four or more such residues, forming two or more π-π stacking pairs at different locations along the peptide.

The two α,α-disubstituted amino acid residues are located at residue positions i and i+x, wherein x is an integer. In some particularly preferred embodiments, the two α,α-disubstituted amino acid residues are located at residue positions i and i+x, wherein x is 3, 4, 6, 7, 8, 9, 10, 11 or 12. For example, the residues may be located at positions i and i+3 or i and i+4; in such embodiments, the residues are separated by a single turn of the α-helix. In some embodiments, the residues may be located at positions i and i+6, i and i+7 or i and i+8; in such embodiments, the residues are separated by two turns of the α-helix. In some embodiments, the residues may be located approximately 3 turns of the α-helix apart, for example at positions i and i+9, i+10, i+11 or i+12.

In particularly preferred embodiments, the α,α-disubstituted amino acids are located at positions i and i+x, wherein x is 3, 4, 6, 7, or 8, i.e. the α,α-disubstituted amino acids are located one or two α-helical turns apart. In further particularly preferred embodiments, the α,α-disubstituted amino acids are located at positions i and i+x, wherein x is 3 or 4, i.e. the α,α-disubstituted amino acids a single π-helical turn apart.

The relative locations of the α,α-disubstituted amino acid residues enable the aromatic groups to be in close proximity on the same face of the α-helix and to form t-t interactions, to stabilise the π-helical structure.

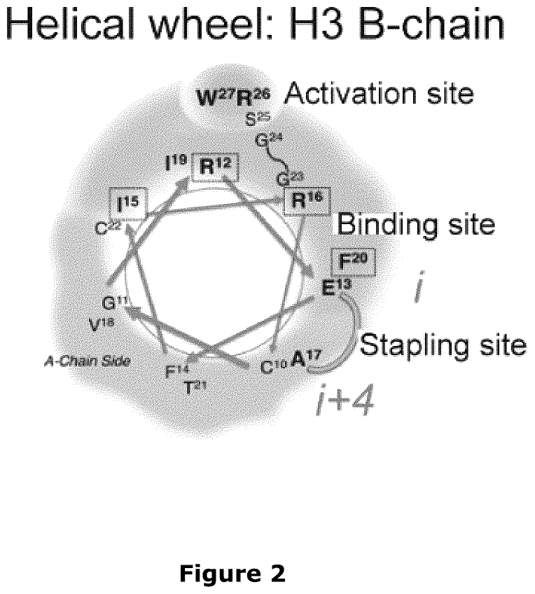

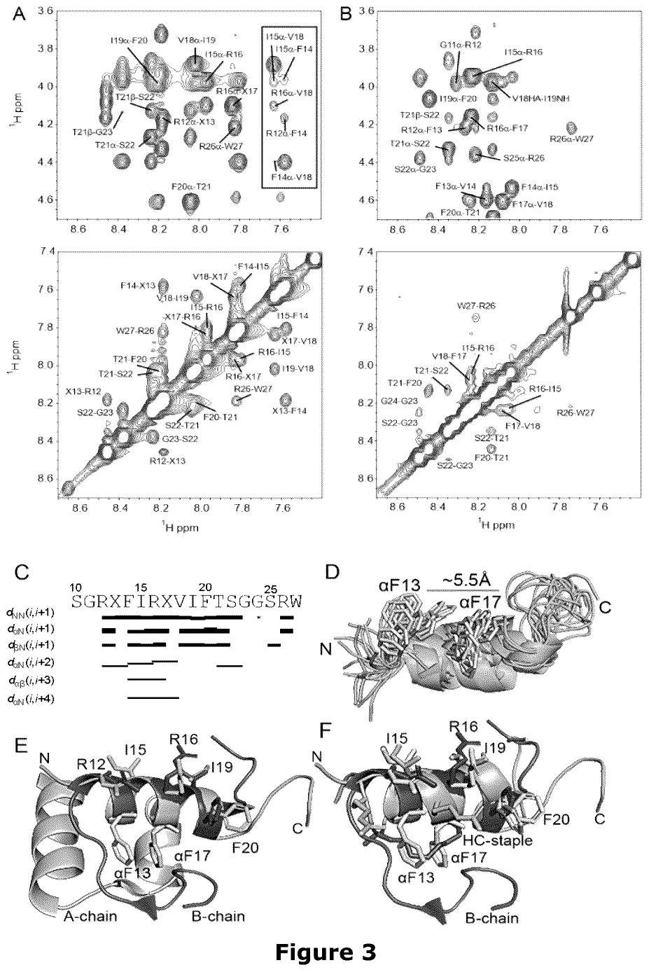

The present inventors have found that, in the case of H3 relaxin (as described in the Examples below), the helical B-chain comprises two faces of the helix, with the active site exposed on one face and the predominantly hydrophobic residues that interact with the A-chain in the native structure exposed on the second face (FIG. 2). The phenyl rings of the αF13 and αF17 (FIG. 3E) in the π-stapled peptide appeared to be in close proximity and formed some weak π-π interaction (not ‘locked’), which resulted in the stabilisation of the helical structure in that region of the peptide. The high resolution, 3-dimensional structure of the π-π stapled peptide, shows that the helical structure adopted by the πH3B peptide is almost identical to the structure of the native B-chain of H3 relaxin (FIG. 3E). While the most critical residues in the binding motif RXXIRXXXF are located on the surface of the helical structure of πH3B, the π-π stabilised residues (αF13/αF17 aromatic rings are within 5 Å, FIG. 3E) are located on the opposite surface of the peptide. As shown in FIG. 3E, the helical structure within πH3B overlays onto the B-chain helix of H3 relaxin with an RMSD of ~1 Å.

The α,α-disubstituted amino acid residues comprise an alkyl, cycloalkyl or alkenyl group as an α-substituent. In particular embodiments, the α,α-disubstituted amino acid may comprise a C₁₋₈ alkyl, cycloalkyl or alkenyl group as a substituent, for example a C₁₋₆ alkyl, cycloalkyl or alkenyl group, for example a C₁₋₄ alkyl, cycloalkyl or alkenyl group. As discussed herein, it is thought that the presence of such a substituent places steric constraints on the peptide backbone that favours helical dihedral angles and thus α-helix formation. Said alkyl or alkenyl substituents may be straight or branched. Said substituents may themselves be optionally substituted, for example substituted with one or more of halogen, cyano, amine, nitro, (C₁-C₄)-alkyl, trifluoromethyl, (C₁-C₄)-alkoxy and/or trifluoromethoxy.

In particularly preferred embodiments, the α-substituent is a methyl group.

The α,α-disubstituted amino acid residues also comprise, as a separate α-substituent, an aromatic group. Said substituent may contain any aromatic ring capable of π-π stacking. π-π stacking is well understood and familiar to a person skilled in the art, and, without wishing to be bound by theory, refers to attractive, noncovalent pi interactions (orbital overlap) between the pi bonds of aromatic rings. π-π stacking may also be referred to as hydrophobic π-π stacking. Possible aromatic groups may include a single aromatic ring or a multicyclic aromatic ring system, and may include hydrocarbon or heterocyclic groups. Exemplary aromatic rings which may be present in the α-substituent include, but are not limited to, phenyl, indenyl, naphthyl, biphenyl, anthracenyl, phenantherenyl, pyridinyl, pyrazinyl, pyrimidinyl, purinyl, imidazyl, furanyl, thiophenyl and pyrryl rings. Said aromatic rings may be optionally substituted with one or more substituents, for example halogen; cyano; nitro; amino; aminoalkyl; (C₁-C₈)-alkyl or alkenyl, which for their part may optionally be mono- or polysubstituted by one or more halogens; (C₃-C₇)-cycloalkyl groups; (C₁-C₅)-alkoxy; and —C(═NH)NH₂.

In particularly preferred embodiments, the α-substituent comprises a chain, for example an alkyl chain, of one or more atoms linking the aromatic group to the α-carbon of the amino acid; that is, in particularly preferred embodiments, the α-substituent is an arylalkylene group. In particularly preferred embodiments, the α-substituent is a benzyl (—CH₂-Ph) group.

In some embodiments, the α,α-disubstituted amino acids are located one α-helical turn apart, for example at residue positions i and i+x, wherein x is 3 or 4, and the α-substituent of one or both residues is an arylmethylene group, that is, the chain is 1 carbon in length.

The two α,α-disubstituted amino acid residues may be the same or different.

In some embodiments, at least one of the α,α-disubstituted amino acids is α-methyl-L-phenylalanine. In some embodiments, both of the α,α-disubstituted amino acids are α-methyl-L-phenylalanine.

In some embodiments, the α-substituent comprises a longer chain connecting aromatic group and the α-carbon of the amino acid, for example an arylalkylene group with a longer alkylene group. For example, the α-substituent may comprise a carbon chain of 2, 3, 4, 5 or 6 carbon atoms. Such α-substituents may facilitate π-π stacking between aromatic rings of α,α-disubstituted amino acid residues which are located further than one π-helical turn apart, for example which are located at positions i and i+x, where x>4, for example i and i+6, i and i+7 or i and i+8.

The present disclosure further relates to pharmaceutical compositions comprising a peptide of the present disclosure and a pharmaceutically acceptable carrier, excipient, diluent, vehicle, and/or adjuvant.

As used herein, the terms “pharmaceutically acceptable carrier”, “pharmaceutically acceptable delivery vehicle” and the like typically mean a biocompatible composition that is capable of being administered to a subject in need thereof, typically in combination with a prophylactic or therapeutic agent, with no or negligible adverse side effects to that subject. The carriers, excipients, diluents, vehicles and adjuvants must be “acceptable” in terms of being compatible with the other ingredients of the formulation, and not deleterious to the recipient thereof.

The pharmaceutical compositions of the present disclosure may be administered by standard routes.

In some embodiments, the pharmaceutical composition of the present disclosure is administered intravenously. For administration as an injectable solution or suspension, non-toxic parenterally acceptable diluents or carriers can include, Ringer's solution, isotonic saline, phosphate buffered saline, ethanol and 1, 2-propylene glycol. Further pharmaceutically acceptable carriers, excipients, diluents, vehicles, and/or adjuvants will be familiar to a person skilled in the art and may be selected depending on various factors such as the identity of the peptide, the subject, the intended use and the intended administration route.

In some embodiments, the pharmaceutical composition of the present disclosure is administered orally.

As discussed above, peptides and proteins of the present disclosure find use as minimised mimetics of larger peptides and proteins, such as naturally occurring peptides or proteins. As such, the peptides of the present disclosure find use in a number of applications, including therapeutic applications in which the natural occurring peptides or proteins on which they are based find use.

Accordingly, the present disclosure relates to peptides of the present disclosure for use in therapy, in particular in treating or preventing a condition. The present disclosure further relates to methods of treating or preventing a condition in a subject, comprising administering an effective amount of a peptide as disclosed herein to a subject in need thereof. The condition may be a disorder as described elsewhere herein. The present disclosure also relates to use of a peptide as described herein in the manufacture of a medicament for the treatment or prevention of a condition, such as a disorder described elsewhere herein.

The present disclosure relates to peptides of the present disclosure for use in treating or preventing a condition associated with RXFP activity. The present disclosure also relates to methods of treating or preventing a condition associated with RXFP activity in a subject, comprising administrating to a subject in need thereof an effective amount of a peptide as disclosed herein. The present disclosure also relates to a use of a peptide as described herein in the manufacture of a medicament for the treatment of prevention of a condition associated with RXFP activity. Any peptide of the present disclosure may find use in the treatment and/or prevention of a condition associated with RXFP activity. In some embodiments, the condition is associated with RXFP1, RXFP2, RXFP3 or RXFP4 activity. In some embodiments, the condition is associated with RXFP1. In some embodiments, the condition is associated with RXFP2. In some embodiments, the condition is associated with RXFP3. In some embodiments, the condition is associated with RXFP4. In some embodiments, the condition is associated with RXFP3 or RXFP4 activity. Accordingly, the present disclosure provides for a method of treating or preventing a condition associated with RXFP activity, preferably RXFP3 or RXFP4 activity, comprising administering to a subject in need thereof an effective amount of a peptide of the present disclosure. The present disclosure also provides for a use of a peptide of the present disclosure in the manufacture of a medicament for the treatment or prevention of a condition associated with RXFP activity, preferably RXFP3 or RXFP4 activity.

Accordingly, the present disclosure also relates to methods of treating or preventing a condition associated with RXFP3 activity in a subject, comprising administrating to a subject in need thereof an effective amount of a peptide as disclosed herein. The present disclosure also relates to uses of a peptide as described herein in the manufacture of a medicament for the treatment of prevention of a condition associated with RXFP3 activity. In some embodiments, the peptide is selective for the RXFP3 receptor. In a further embodiment, the peptide is an agonist of the RXFP3 receptor. In another embodiment, the peptide is a selective agonist of the RXFP3 receptor. In a further embodiment, the peptide is an antagonist of the RXFP3 receptor. In a further embodiment, the peptide is a selective antagonist of the RXFP3 receptor. In some embodiments, the peptide is an analogue of the B chain of insulin-like peptide 7 or a fragment thereof. In some embodiments, the condition associated with RXFP3 activity is a neuropsychiatric disorder, an eating disorder, weight loss, weight gain, obesity or pain. In some embodiments, the condition associated with RXFP3 activity is an eating disorder, weight loss, weight gain, or obesity. In some embodiments, the condition associated with RXFP3 activity is obesity. In some embodiments, the condition associated with RXFP3 activity is pain. Peptides of the present disclosure which are selective for RXFP3 and/or are analogues of the B chain of insulin-like peptide 7 or a fragment thereof, may find particular use in the treatment and/or prevention of such disorders. Accordingly, in some embodiments the condition is associated with RXFP3 activity, preferably wherein the peptide is an analogue of the B chain of insulin-like peptide 7. Peptides of the present disclosure which are agonists of the RXFP3 receptor may find particular use in the treatment and/or prevention of an eating disorder, weight loss, pain or a neuropsychiatric disorder. Peptides of the present disclosure which are antagonists of the RXFP3 receptor may find particular use in the treatment and/or prevention of an eating disorder, weight gain, or obesity.

By way of example, research suggests INSL7 (H3 relaxin) has potential application for the treatment of neuropsychiatric disorders such as anxiety and depression (Smith, C. M et al. Front Pharmacol 2014, 5, 46. 24711793). The global economy loses about $1 trillion every year in productivity due to depression and anxiety. As such, peptides of the present disclosure which are analogues of the B chain of H3 relaxin may find use in the treatment and/or prevention of such disorders.

Accordingly, the present disclosure relates to peptides according to the present disclosure for use in the treatment or prevention of a neuropsychiatric disorder. The present disclosure also provides for a method of treating or preventing a neuropsychiatric disorder, comprising administering an effective amount of a peptide disclosed herein to a subject in need thereof. The present disclosure also provides for a use of a peptide disclosed herein the manufacture of a medicament for the treatment or prevention of a neuropsychiatric disorder. In some embodiments, the neuropsychiatric disorder is selected from depression and anxiety. In some embodiments, the neuropsychiatric disorder is depression. In some embodiments, the neuropsychiatric disorder is anxiety.

The present disclosure also relates to peptides according to the present disclosure for use in the treatment or prevention of an eating disorder. The present disclosure also provides for a method of treating or preventing an eating disorder, comprising administering an effective amount to a subject in need thereof of a peptide disclosed herein. The present disclosure also provides for a use of a peptide disclosed herein the manufacture of a medicament for the treatment or prevention of an eating disorder. In some embodiments, the eating disorder is an eating disorder associated with a psychological or psychiatric disorder characterised by abnormal or disturbed eating habits. In some embodiments, the eating disorder is selected from pica, anorexia nervosa, bulimia nervosa, rumination disorder, avoidant/restrictive food intake disorder, binge-eating disorder, stress-related eating disorders, food addiction, eating disorders associated with taking drugs (e.g. anxiolytic drugs, antipsychotic drugs or antidepressant drugs), or a combination thereof.

The present disclosure also relates to peptides according to the present disclosure for use in the treatment or prevention of weight loss. The present disclosure also provides for a method of treating or preventing weight loss, comprising administering an effective amount of a peptide disclosed herein to a subject in need thereof. The present disclosure also provides for a use of a peptide disclosed herein the manufacture of a medicament for the treatment or prevention of weight loss. It will be understood that such treatment may encompass the increasing, maintaining gain of, or delaying the loss of weight. In some embodiments, the weight loss is selected from age-related weight loss, stress-related weight loss, weight loss associated with taking drugs (e.g. anxiolytic drugs, antipsychotic drugs, antidepressant drugs or chemotherapeutics), or weight loss associated with a psychological or psychiatric disorder. In some embodiments, the weight loss is selected from anxiety-related weight loss, depression-related weight loss, or stress-related weight loss.

The present disclosure also relates to peptides according to the present disclosure for use in the treatment or prevention of obesity. The present disclosure also provides for a method of treating or preventing obesity, comprising administering an effective amount of a peptide disclosed herein to a subject in need thereof. The present disclosure also provides for a use of a peptide disclosed herein the manufacture of a medicament for the treatment or prevention of obesity. As used herein, the term “obesity” refers to an excess of adipose tissue in the body. There is no sharp distinction between normal individuals, overweight individuals and those suffering from obesity, but the health risk accompanying obesity is presumed to rise continuously as the extent of adipose tissue and/or “fatness” increases. Individuals with a Body Mass Index (BMI), above a value of 25 and more particularly above 30, are preferably regarded as suffering from obesity. BMI refers to the body weight of a subject in kilograms divided by the square of the subject's height in meters.

In some embodiments, the obesity is obesity associated with metabolic syndrome, hypertension, osteoarthritis, diabetes (e.g. type II diabetes), complications of diabetes (e.g. diabetic retinopathy, diabetic neuropathy, diabetic nephropathy, insulin resistance), pathological glucose tolerance, encephalorrhagia, heart diseases, cardiac insufficiency, arteriosclerosis, arthritis, gonitis, stroke or dyslipidaemia. In some embodiments, the obesity is obesity associated with metabolic syndrome, diabetes or dyslipidaemia.

The present disclosure also relates to peptides according to the present disclosure for use in the treatment or prevention of weight gain. The present disclosure also provides for a method of treating or preventing a weight gain, comprising administering an effective amount of a peptide disclosed herein to a subject in need thereof. The present disclosure also provides for a use of a peptide disclosed herein the manufacture of a medicament for the treatment or prevention of weight gain. It will be understood that such treatment may encompass the reducing, maintaining loss of, or delaying the increase of weight. It will be understood that “weight gain” refers to an increase in weight of a subject, compared to that subject's weight at a previous point in time, or compared to a reference weight. In some embodiments, the reference weight corresponds to about 25 BMI (Body Mass Index). In some embodiments, the weight gain is selected from age-related weight gain, stress-related weight gain, weight gain associated with taking drugs (e.g. anxiolytic drugs, antipsychotic drugs, antidepressant drugs or chemotherapeutics), or weight gain associated with a psychological or psychiatric disorder. In some embodiments, the weight gain is selected from anxiety-related weight gain, depression-related weight gain, or stress-related weight gain.

The present disclosure also relates to peptides according to the present disclosure for use in reducing weight or weight gain. The present disclosure also provides for a method of reducing weight or weight gain, comprising administering an effective amount of a peptide disclosed herein to a subject in need thereof. The present disclosure also provides for a use of a peptide disclosed herein the manufacture of a medicament for reducing weight or weight gain.

In some embodiments, the treatment and/or prevention of weight gain or obesity, or the reduction in weight or weight gain, provides a reduction in a subject's BMI of about 1, 2, 3, 4, 5, 6, 7, 8, 9 or 10. In some embodiments, treatment or prevention of weight gain or obesity, or the reduction in weight or weight gain, provides a reduction in a subject's BMI of about 5%, 10%, 15%, 20%, 25%, 30%, 35%, 40%, 45% or 50%. In some embodiments, the treatment or prevention of weight gain or obesity, or the reduction in weight or weight gain, results in a lowering of the subject's BMI to less than about 60, 55, 50, 45, 40, 35, 30, 25, 20 or 15. In some embodiments, the treatment or prevention of weight gain or obesity, or the reduction in weight or weight gain, results in a lowering of the subject's BMI to less than about 30. In some embodiments, the treatment or prevention of weight gain or obesity, or the reduction in weight or weight gain, results in a lowering of the subject's BMI to less than about 25. In some embodiments, the treatment or prevention of weight gain or obesity, or the reduction in weight or weight gain, results in a lowering of the subject's BMI to an amount between about 15 and about 30, or between about 18 and about 25. In some embodiments, treatment or prevention of weight gain or obesity, or the reduction in weight or weight gain, provides a reduction in a subject's weight (in kg) of less than 1, 5, 10, 15, 20, 25, 30, 35, 40, 45, 50, 55, 60, 65 or 70. In some embodiments, treatment or prevention of weight gain or obesity, or the reduction in weight or weight gain, provides a reduction in a subject's weight (in kg) of more than 1, 5, 10, 15, 20, 25, 30, 35, 40, 45, 50, 55, 60, 65 or 70. In some embodiments, treatment or prevention of weight gain or obesity, or the reduction in weight or weight gain, provides a reduction in a subject's weight in range provided by any of the previously described upper and/or lower amounts, for example, in some embodiments, treatment or prevention of weight gain or obesity, or the reduction in weight or weight gain, provides a reduction in a subject's weight (in kg) of between about 1 and about 70, between about 5 and about 30, between about 5 and about 20, or between about 5 and about 10. It will be understood that a reduction in weight or BMI, may be given in reference to the subject's weight or BMI (respectively) at a previous point in time, or compared to a reference weight or BMI. In some embodiments, the reference weight corresponds to about 25 BMI (Body Mass Index), or the equivalent weight thereof of the subject.

The present disclosure also relates to peptides according to the present disclosure for use in the treatment, prevention or management of pain. The present disclosure also provides for a method of treating, preventing or managing pain, comprising administering an effective amount of a peptide disclosed herein to a subject in need thereof. The present disclosure also provides for a use of a peptide disclosed herein the manufacture of a medicament for the treatment, prevention or management of pain. In some embodiments, the pain is nerve pain. In some embodiments, the pain is muscle pain. In some embodiments, the pain is neuropathic pain. In some embodiments, the pain is joint pain. In some embodiments, the pain is arthritis-induced pain. In some embodiments, the pain is chronic pain.

The present disclosure also relates to a method of treating or preventing a condition associated with RXFP4 activity in a subject, comprising administrating to a subject in need thereof an effective amount of a peptide as disclosed herein. The present disclosure also relates to a use of a peptide as described herein in the manufacture of a medicament for the treatment or prevention of a condition associated with RXFP4 activity. In some embodiments, the peptide is selective for the RXFP4 receptor. In a further embodiment, the peptide is an agonist of the RXFP4 receptor. In another embodiment, the peptide is a selective agonist of the RXFP4 receptor. In some embodiments, the peptide is an analogue of the B chain of insulin-like peptide 5 or a fragment thereof.

INSL5 finds potential application in the treatment of colon motility disorders, including constipation and diarrhoea. As such, peptides of the present disclosure which are analogues of the B chain of INSL5 may find use in the treatment and/or prevention of such disorders. Further, peptides of the present disclosure, e.g. where the peptide is selective for the RXFP4 receptor, and/or is an agonist of the RXFP4 receptor, and/or is an analogue of the B chain of insulin-like peptide 5 or a fragment thereof, may find particular use in the treatment and/or prevention of a colon motility disorder. Accordingly, in some embodiments, the condition is associated with RXFP4 activity, and the peptide is an analogue of the B chain of insulin-like peptide 5 or a fragment thereof.

Accordingly, the present disclosure relates to peptides according to the present disclosure for use in the treatment or prevention of a colon motility disorder. The present disclosure also provides for a method of treating or preventing a colon motility disorder, comprising administering an effective amount of a peptide disclosed herein to a subject in need thereof. The present disclosure also provides for a use of a peptide disclosed herein the manufacture of a medicament for the treatment or prevention of a colon motility disorder. In some embodiments, the colon motility disorder is constipation or diarrhoea. In some embodiments, the colon motility disorder is diarrhoea. In some embodiments, the colon motility disorder is constipation. In some embodiments, the constipation is chronic constipation, constipation associated with spinal cord injury, constipation associated with use of opiate pain killers, post-surgical or post-operative constipation, constipation associated with neuropathic disorders, constipation associated with Parkinson's disease, constipation associated with irritable bowel syndrome, or idiopathic constipation. In some embodiments, the constipation is chronic constipation. In some embodiments, the constipation is constipation associated with spinal cord injury. In some embodiments, the constipation is constipation associated with use of opiate pain killers. In some embodiments, the constipation is post-surgical or post-operative constipation. In some embodiments, the constipation is constipation associated with neuropathic disorders. In some embodiments, the constipation is constipation associated with Parkinson's disease. In some embodiments, the constipation is constipation associated with irritable bowel syndrome. In some embodiments, the constipation is idiopathic constipation. Peptides of the present disclosure which are agonists of the RXFP4 receptor may find particular use in the treatment and/or prevention of the above disease and conditions.

The present disclosure also relates to a method of treating or preventing a condition associated with RXFP1 activity in a subject, comprising administrating to a subject in need thereof an effective amount of a peptide as disclosed herein. The present disclosure also relates to a use of a peptide as described herein in the manufacture of a medicament for the treatment of prevention of a condition associated with RXFP1 activity. In some embodiments, the peptide is selective for the RXFP1 receptor. In a further embodiment, the peptide is an agonist of the RXFP1 receptor. In another embodiment, the peptide is a selective agonist of the RXFP1 receptor. In a further embodiment, the peptide is an antagonist of the RXFP1 receptor. In a further embodiment, the peptide is a selective antagonist of the RXFP1 receptor. In some embodiments, the peptide is an analogue of the B chain of H2-relaxin or a fragment thereof. In some embodiments, the condition associated with RXFP1 activity is fibrosis or a fibrotic disorder, or cardiovascular disorders. In some embodiments, the condition associated with RXFP1 activity is fibrosis or a fibrotic disorder. In some embodiments, the condition associated with RXFP1 activity is a cardiovascular disorder. Peptides of the present disclosure which are selective for RXFP1 and/or are analogues of the B chain of H2-relaxin or a fragment thereof, may find particular use in the treatment and/or prevention of such disorders.

The present disclosure also relates to peptides according to the present disclosure for use in the treatment or prevention of fibrosis or a fibrotic disorder, or a cardiovascular disorder. The present disclosure also provides for a method of treating or preventing fibrosis or a fibrotic disorder, or cardiovascular disorders, comprising administering an effective amount of a peptide disclosed herein to a subject in need thereof. The present disclosure also provides for a use of a peptide disclosed herein the manufacture of a medicament for the treatment or prevention of fibrosis or a fibrotic disorder, or cardiovascular disorders. For example, peptides of the disclosure may find application in the treatment of renal fibrosis, hepatic fibrosis, pulmonary fibrosis, cardiac fibrosis, coronary artery disease, pancreatitis, inflammation, acute heart failure, microvascular disease, preeclampsia, hypertensive diseases, scleroderma, cervical ripening, and/or fibromyalgia. In one embodiment, the condition is renal fibrosis. In one embodiment, the condition is hepatic fibrosis. In one embodiment, the condition is pulmonary fibrosis. In one embodiment, the condition is cardiac fibrosis. In one embodiment, the condition is coronary artery disease. In one embodiment, the condition is pancreatitis. In one embodiment, the condition is inflammation. In one embodiment, the condition is acute heart failure. In one embodiment, the condition is microvascular disease. In one embodiment, the condition is preeclampsia. In one embodiment, the condition is hypertensive disease. In one embodiment, the condition is scleroderma. In one embodiment, the condition is cervical ripening. In one embodiment, the condition is fibromyalgia. Peptides of the present disclosure which are agonists of the RXFP1 receptor may find particular use in the treatment and/or prevention of the above disorders or conditions, such as fibrosis or a fibrotic disorder, or a cardiovascular disorder. Peptides of the present disclosure which are antagonists of the RXFP1 receptor may find particular use in the treatment and/or prevention of the above disorders or conditions.

The present disclosure also relates to a method of treating or preventing a condition associated with RXFP2 activity in a subject, comprising administrating to a subject in need thereof an effective amount of a peptide as disclosed herein. The present disclosure also relates to a use of a peptide as described herein in the manufacture of a medicament for the treatment of prevention of a condition associated with RXFP2 activity. In some embodiments, the peptide is selective for the RXFP2 receptor. In a further embodiment, the peptide is an agonist of the RXFP2 receptor. In another embodiment, the peptide is a selective agonist of the RXFP2 receptor. In a further embodiment, the peptide is an antagonist of the RXFP2 receptor. In a further embodiment, the peptide is a selective antagonist of the RXFP2 receptor. In some embodiments, the peptide is an analogue of the B chain of insulin-like peptide 3 or a fragment thereof. In some embodiments, the condition associated with RXFP2 activity is a bone disorder, a reproductive disease or disorder, hypogonadism, cryptorchidism, polycystic ovary syndrome (PCOS), cancer, infertility, or an ocular (eye) wound. In some embodiments, the condition associated with RXFP2 activity is a bone disorder. Examples of suitable bone disorder include, but are not limited to, osteoporosis, osteopenia, or osteogenesis imperfecta. In some embodiments, the condition associated with RXFP2 activity is a reproductive disease or disorder. In some embodiments, the condition associated with RXFP2 activity is hypogonadism. In some embodiments, the condition associated with RXFP2 activity is cryptorchidism. In some embodiments, the condition associated with RXFP2 activity is polycystic ovary syndrome (PCOS). In some embodiments, the condition associated with RXFP2 activity is cancer. Examples of suitable cancers include, but are not limited to, testicular cancer, prostate cancer, or thyroid cancer. In some embodiments, the condition associated with RXFP2 activity is infertility. In some embodiments, the condition associated with RXFP2 activity is an ocular (eye) wound. Peptides of the present disclosure which are selective for RXFP2 and/or are analogues of the B chain of insulin-like peptide 3 or a fragment thereof, may find particular use in the treatment and/or prevention of such disorders and conditions. Peptides of the present disclosure which are agonists of the RXFP2 receptor may find particular use in the treatment and/or prevention of the above conditions (e.g. reproductive diseases or disorders). Peptides of the present disclosure which are antagonists of the RXFP2 receptor may find particular use in the treatment and/or prevention of the above conditions (e.g. cancer, such as prostate cancer).

The present disclosure also relates to a method of contraception, comprising administrating to a subject an effective amount of a peptide as disclosed herein. The present disclosure also relates to uses of a peptide as described herein in the manufacture of a medicament for use as a contraceptive. In some embodiments thereof, the peptide is selective for RXFP2. In some embodiments thereof, the peptide is an agonist of RXFP2.

Peptides nociception and galanin (GAL) find potential in the treatment of pain. As such, peptides of the present disclosure which are analogues of said peptides may find use in the treatment and/or prevention of pain.

Accordingly, the present disclosure relates to peptides according to the present disclosure for use in the treatment or prevention of pain.

Neuropeptide Y (NPY) finds potential in the treatment of epilepsy. As such, peptides of the present disclosure which are analogues of NPY may find use in the treatment and/or prevention of epilepsy.

Accordingly, the present disclosure relates to peptides according to the present disclosure for use in the treatment or prevention of epilepsy.

It will be appreciated that features described herein in relation to one aspect of the present disclosure may be readily applied or modified for application to another aspect of the present disclosure. Disclosure of a feature in relation to one aspect includes its disclosure in relation to other aspects.

It will be appreciated by persons skilled in the art that numerous variations and/or modifications may be made to the disclosure without departing from the spirit or scope of the disclosure as broadly described. The present embodiments are, therefore, to be considered in all respects as illustrative and not restrictive.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure will now be further described in greater detail by reference to the following specific examples, which should not be construed as in any way limiting the scope of the disclosure.

### EXAMPLES

### Example 1—Preparation of Stapled Human Insulin-Like Peptide 7 (INSL 7; H3 Relaxin)

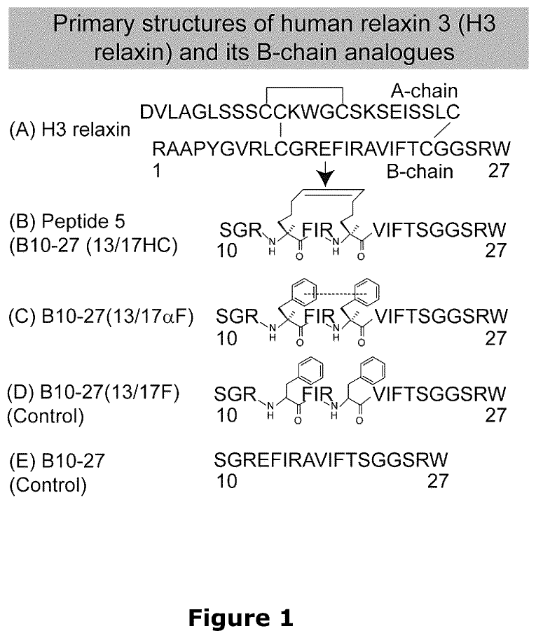

Human insulin-like peptide 7 (INSL7), also known as human relaxin-3 (H3 relaxin) (an insulin-like relaxin family peptide) contains, in its native form, two chains (A and B) and three disulfide bridges (FIG. 1A):

A chain
(SEQ ID NO: 1)
DVLAGLSSSCCKWGCSKSEISSLC
B chain
(SEQ ID NO: 2)
RAAPYGVRLCGREFIRAVIFTCGGSRW

The present inventors hypothesised that, for preparing B-chain minimized mimetics of H3 relaxin, the incorporation of a unique α-disubstituted amino acid, α-methyl-L-phenylalanine (αF), would result in an π-helical structure of the otherwise unstructured linear H3 relaxin B-chain. In particular the present inventors hypothesized that, in addition to the α-methyl group in αF that places steric constraints on the peptide backbone that favours helical dihedral angles, there should be additional hydrophobic or π-π interactions between the two aromatic phenyl rings oriented towards each other on the same side of the peptide, leading to a non-covalently stapled helical peptide. Accordingly, B-chain peptide variants were prepared as follows: i) a hydrocarbon-(HC) stapled single-B-chain peptide (FIG. 1B; ‘Peptide 5’ (SEQ ID NO 3)), stapled at positions 13 and 17; ii) peptide B10-27(13/17αF), containing two αF residues at positions 13 and 17 (FIG. 1C (SEQ ID NO 4)); iii) a control peptide B10-27(13/17F) that contains L-phenylalanine at positions 13 and 17 (FIG. 1D (SEQ ID NO 5)); and iv) native B10-27 (FIG. 1E (SEQ ID NO 6)) (the residue numberings of which are inherited from those of the B chain of native INSL7, SEQ ID NO 2):

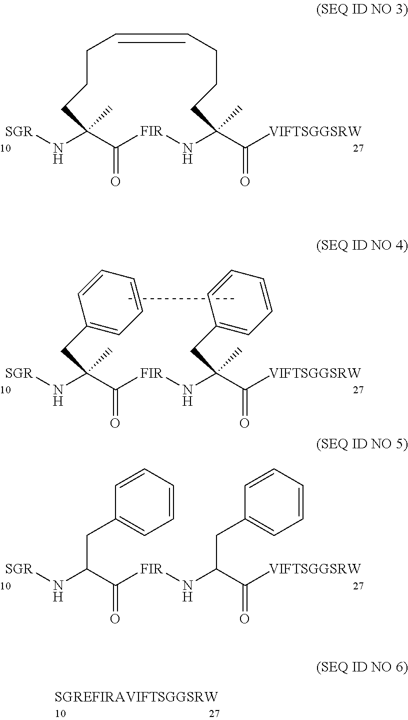

All single-chain peptides were synthesized using Fmoc solid phase synthesis on a microwave-assisted peptide synthesizer (CEM Liberty, Mathew, USA) or manually using Fmoc protected L-α-amino acids. Peptide chains were assembled on preloaded resins. All side chain-protecting groups of amino acids were TFA-labile. The peptides were synthesized on either 0.1 or 0.2 mmol scale using instrument default protocols or manually with a 4-fold molar excess of Fmoc-protected amino acids activated by 4-fold excess of HCTU/HATU in the presence of excess (6-fold) DIEA. Nα-Fmoc protecting groups were removed by treating the resin attached peptide with piperidine (20% v/v) in DMF. Using the microwave synthesizer, the coupling and deprotection were conducted at 75° C. using 25 W microwave power for 5 min and 60 W microwave power for 5 or 3 min, respectively. For manual synthesis, the coupling and deprotection were carried out for 60 min and 5 min three times, respectively. Peptide 5 corresponding to SEQ ID NO:3 was prepared as described in Hojo, K et al., Development of a single-chain peptide agonist of the relaxin-3 receptor using hydrocarbon stapling. J Med Chem 2016, 59, 7445-7456. The synthetic methods for RXFP3 agonists, R3/151, A22 and Peptide 53 have previously been reported (Haugaard-Jonsson, L. M. et al, Structure of the R3/I5 chimeric relaxin peptide, a selective GPCR135 and GPCR142 agonist. J Biol Chem 2008, 283 (35), 23811-8; Shabanpoor, F. et al., Minimization of human relaxin-3 leading to high-affinity analogues with increased selectivity for relaxin-family peptide 3 receptor (RXFP3) over RXFP1. J Med Chem 2012, 55, 1671-1681; Hojo, K. et al., Development of a single-chain peptide agonist of the relaxin-3 receptor using hydrocarbon stapling. J Med Chem 2016, 59, 7445-7456).

After completion of solid phase synthesis, peptides were cleaved using a cleavage cocktail of trifluoroacetic acid (TFA):TIPS water:anisol (94:1:2:3) for 2 hours. Cleavage solution was filtered and evaporated under nitrogen. The cleaved peptide was precipitated in cold ether and centrifuged for 5 min; this step was repeated at least four times.

Analytical RP-HPLC analysis of the purified peptides was performed with Waters RP-HPLC systems. Empower software was used for data collection, monitoring, and analysis. The RP-HPLC profiles were acquired using a Phenomenex Gemini C18 analytical column (4.6×250 mm, pore size 110 A, particle size 5 μm), at a constant flow rate of 1.5 mL/min, in a gradient mode with buffer A, 0.10% TFA in water, and buffer B, 0.1% TFA in acetonitrile, monitoring at a wavelength of 214 nm, which is characteristic for the amide bond. All HPLC purifications were performed using a Phenomenex C18 preparative column (21.2×150 mm), at a constant flow rate of 10 mL/min, in a gradient mode with eluent A, 0.1% TFA in water, and eluent B, 0.1% TFA in acetonitrile.

Peptide characterization was conducted on a Bruker Ultraflex II instrument (Bruker Daltonics, Bremen, Germany) MALDI-TOF MS (matrix-assisted laser desorption ionization time-of-flight mass spectrometry) and SHIMADZU MALDI-8020. Sinapinic acid and DHB in 0.1% TFA in ACN/water (7:3) was used as a matrix.

Peptide content was determined using Direct Detect® assay-free sample cards and the Direct Detect® spectrometer. All measurements were performed using 2 μL of the sample solution.

9-Fluoroenylmethoxycarbonyl (Fmoc), and O-(1H-6-chlorobenzotriazole-1-yl)-1,1,3,3-tetramethyluronium hexafluorophosphate (HCTU)/1-[Bis(dimethylamino)methylene]-1H-1,2,3-triazolo[4,5-b]pyridinium 3-oxide hexafluorophosphate (HATU) were purchased from GL Biochem (Shanghai, China). Fmoc-Trp (Boc) TentaGel S PHB resin (substitution 0.24 mmol/g), trifluoroacetic acid (TFA), and diisopropylethelene amine (DIEA) were obtained from Auspep (Melbourne, Australia). Acetonitrile, dichloromethane, piperidine (PPD), diethyl ether, N,N′-dimethylformamide (DMF) and methanol were purchased from Merck (Melbourne, Australia). Fmoc-α-methyl-L-phenylalanine was purchased from CHEM IMPEX INTERNATIONAL Inc. Triisopropylsilane (TIPS) anisol, sinapinic acid (3,5-dimethyl-4-hydroxycinnamic acid), DHB (2,5-dihydroxybenzoic acid) and human serum were purchased from Sigma-Aldrich (Sydney, Australia).

MALDI-TOF MS, HPLC purity and yield of H3 relaxin-based peptides are given in Table 1 below.

TABLE 1

| | MALDI TOF MS | | HPLC | Purity | Yield |
|---|---|---|---|---|---|
| B-Chain Analogues | Observed¹ | Calculated | t_{R} (min) | (%) | (%) |
| H3B10-27 (SEQ ID | 2029.436 | 2026.264 | 10.927 | 99 | 50 |
| NO 6) | | | | | |
| H3B10-27(13/17F) | 2121.172 | 2120.422 | 10.717 | >99 | 67 |
| (SEQ ID NO 5) | | | | | |
| H3B10-27(13/17αF) | 2150.729 | 2148.991 | 13.682 | >99 | 60 |
| (SEQ ID NO 4) | | | | | |

¹Observed molecular weights were determined by MALDI-TOF MS of purified peptides

Chemical synthesis of H3 relaxin and HC-stapled Peptide 5 is tedious and laborious compared with H3B10-27(13/17αF) (Smith, C. M. et al., Relaxin-3/RXFP3 networks: an emerging target for the treatment of depression and other neuropsychiatric diseases? Front Pharmacol 2014, 5, 46). The B-chain-only analogue containing the non-covalent staple developed by the present inventors (18 residues, yield 60%) was very high yielding compared to H3 relaxin (51 residues, yield less than 2% starting from the A-chain, 5.7% starting from the B-chain; Bathgate, R. A. et al., Relaxin-3: improved synthesis strategy and demonstration of its high-affinity interaction with the relaxin receptor LGR7 both in vitro and in vivo. Biochemistry 2006, 45, 1043-1053) and Peptide 5 (~10%). Whilst the HC-stapled Peptide 5 has an unknown (Cis or Trans) conformation and is insoluble in water, H3B10-27(13/17αF) is a single species and water soluble.

The inventors hypothesized that both α-methyl groups and phenyl rings of the αF residues are essential to induce π-helical conformation of the peptide and thus critical to retain their biological function. Solution NMR studies were on both the target, H3 B10-27(13/17αF) (SEQ ID NO:4) and one of the control peptides, H3 B10-27(13/17F) (SEQ ID NO:5) to confirm the presence of an π-helical conformation in H3 B10-27(13/17αF) similar to the native H3 relaxin B-chain.

All NMR experiments were routinely collected at 15° C. on a 700 MHz Bruker Advance HDIII spectrometer equipped with triple resonance cryoprobe. Peptide (2 mg) was dissolved in 0.5 mL of 90% H2O-10% D2O imidazole-d4 buffer at ~pH 6.8. Two-dimensional data sets including a homonuclear proton TOCSY (1024×256 complex points) with a mixing time of 80 ms, 1H-1H NOESY (1024×256 complex points) with a mixing time of 200 ms and a 1H-15N HSQC (1024×256 complex points) at natural abundance were recorded. Spectra were processed using NMRPipe and typically Fourier-transformed after applying Lorentz-to-Gauss window functions in the direct dimension and cosine bells in the indirect dimensions. NMR data were analysed using SPARKY11-15.

For H3 B10-27 (13/17αF) peptide structure determination, ~60 sequential (i, i+1 and 1−i+2 or greater) cross peaks in the 1H-1H NOESY spectrum were picked, assigned and integrated using Sparky and translated into interproton distances using CYANA. A pool of 100 three-dimensional structures were generated using torsion angle dynamics protocol (~18 torsion angles) and only 20 structures with the lowest CYANA target function (0.08+0.01 Å2) were selected to best represent the structure of the target peptide.

Both peptides produced good quality NMR data with narrow line widths, which led to a near-complete resonance assignment using standard two-dimensional sequential assignment strategies. FIGS. 3A and B illustrate regions of the 2D NOESY spectra for the target H3 B10-27(13/17αF) (SEQ ID NO 4) and the control peptide H3 B10-27(13/17F) (SEQ ID NO 5), respectively. For the target peptide, H3 B10-27(13/17αF), strong HNᵢ-HNᵢ₊₁ sequential signals were observed throughout the residues ranging from Arg12-Gly23. Importantly, several short-range NOES, Hαᵢ-HNᵢ₊₂, Hαᵢ-HNᵢ₊₄, and Haᵢ-Hpᵢ₊₃ (FIGS. 3A and C) were also identified within the same region, supporting the presence of a helical structure; while for the control peptide the inventors observed HNᵢ-HNᵢ₊₁ sequential NOEs, the lack of short-range NOEs suggesting this peptide comprises an unstable and transient helical structure (FIG. 3B). Using structural restraints from the NMR data, the structure of H3 B10-27(13/17αF) was calculated.

FIG. 3D illustrates a family of 20 low-energy structures representing the solution conformation of the target peptide H3 B10-27(13/α17F), which highlights a well-defined helical structure stretching from the αF13 to F20. This structure displays the two aromatic phenyl rings of αF13 and αF17 oriented towards each other on the same side of the peptide and are within an average of 5.5 Δ distance, which would favor formation of π-π or hydrophobic interactions which may help stabilize a helical structure. Literature reports suggest that, in the specific ring arrangements and angles, the π-π stacking interactions are possible when the distance is within 7 Δ (Kruszynski, R.; Sieranski, T., Can stacking interactions exist beyond the commonly accepted limits? Crystal Growth & Design 2016, 16, 587-595). However, as such interactions are also possible in the control peptide with two phenyl moieties, they are clearly insufficient to stabilize the helix. These data support the notion that α-methyl substitutions are essential. Without wishing to be bound by theory, it is thought that the α-methyl substitutions restrict peptide flexibility and put a steric constraint on the peptide backbone that favors helix formation. As shown in FIG. 3E, the helical structure within the target peptide overlays onto the B-chain helix of H3 relaxin with an RMSD of ~1 Å, highlighting the key receptor binding residues such as Arg12, Ile15, Arg16 and Phe20, exposed on the surface of the helix. The target peptide is also structurally very similar to the HC-stapled Peptide 5 (SEQ ID NO 3), as shown in FIG. 3F (Hojo, K. et al., Development of a single-chain peptide agonist of the relaxin-3 receptor using hydrocarbon stapling. J Med Chem 2016, 59, 7445-7456).

Together, this validates the formation of a helical structure in the target peptide containing α-methyl substitution of Phe residues at position 13 and 17, which is very similar to the native H3 relaxin peptide and HC-stapled Peptide 5, supporting the efficient binding and activity observed for the target peptide against RXFP3 discussed below.

### Example 2—In Vitro Stability of INSL7 Analogues

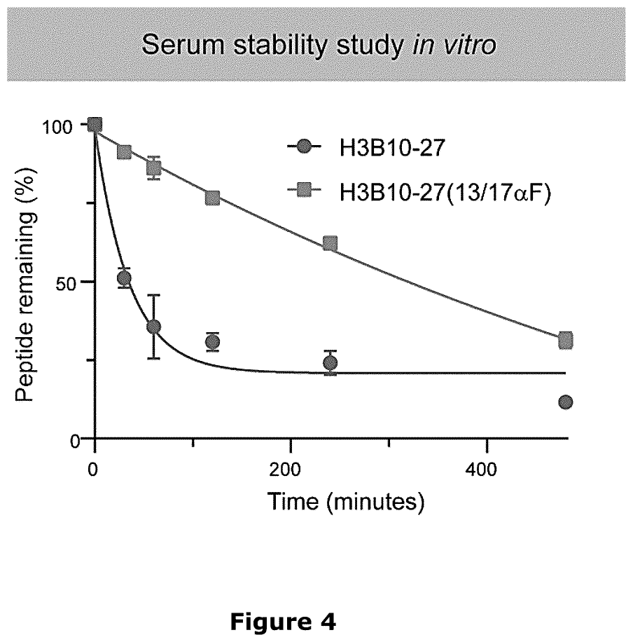

Unstructured native peptides are more easily accessible to proteases and thus less stable in serum than structured peptides. Therefore, the in vitro serum stability of the novel agonist H3B10-27(13/17αF) (SEQ ID NO 4) was examined and compared with the control H3B10-27 (SEQ ID NO 6) for 8 h at 37° C. (FIG. 4).

Human serum (400 μl) was mixed with 200 μg peptide dissolved in 100 μl of water at 37° C. Eighty (80) μl of sample was removed at various time points (0 min, 30 min, 60 min, 120 min, 240 min, and 480 min), and 160 μl of acetonitrile added to precipitate plasma proteins. The solution was then centrifuged at 12000 rpm for 15 min at 4° C. 200 μl of supernatant was taken and mixed with 240 μl of milli-Q water and each sample analysed by RP-HPLC (2×80 μL injection) by measuring the area under the peak at appropriate retention times compared to the peak area of the peptide at time zero (100%). Data analysis (n=3, duplicate) was performed using a non-linear fit one-phase decay model in GraphPad Prism 9.

The agonist H3B10-27(13/17αF) was ~15-fold more stable in human serum (half-life ~300 min) than the control peptide (half-life ~20 min) supporting the structural data that H3B10-27(13/17αF) is more folded than the control peptide. The two unnatural amino acids (αF) cannot be recognized by proteases and thus should also contribute to the improved stability in serum.

### Example 3—Cell-Based Assays for Binding and Potency

### 3.1—RXFP3 and RXFP4 Binding Assays

The purified H3 relaxin-based analogues produced in Example 1 were first tested in cell-based assays for RXFP3 binding affinity. An Eu-H3B1-22R competition binding assay was conducted in CHO-K1-RXFP3 cells.

Since H3 relaxin also binds to and activates the INSL5 receptor, RXFP4, the peptide analogues were then tested for RXFP4 binding and activity. An Eu-R3/15 competition binding assay in CHO-K1-RXFP4 cells was carried out.

Chinese hamster ovary CHO-K1 cells stably transfected with RXFP3 (Van der Westhuizen, E. T. et al., Responses of GPCR135 to human gene 3 (H3) relaxin in CHO-K1 cells determined by microphysiometry. Ann N Y Acad Sci 2005, 1041, 332-7) or RXFP4 (Belgi, A. et al., Structure and function relationship of murine insulin-like peptide 5 (INSL5): free C-terminus is essential for RXFP4 receptor binding and activation. Biochemistry 2011, 50 (39), 8352-61) were plated onto pre-coated, poly-L-lysine 96-well view plates at a density of 50000 cells per well. Medium was aspirated and cells were washed with phosphate-buffered saline (PBS) before competition binding assays were performed with 5 nM Eu-H3B1-22R (Haugaard-Kedstrom, L. M. et al., Synthesis and pharmacological characterization of a europium-labelled single-chain antagonist for binding studies of the relaxin-3 receptor RXFP3. Amino Acids 2015, 47 (6), 1267-71. (RXFP3 ligand)) or 5 nM Eu-DTPA-R3/15 (Zhang, X. et al. Human Insulin-like Peptide 5 (INSL5). Identification of a Simplified Version of Two-Chain Analog A13. ACS Med Chem Lett 2020, 11(12), 2455-2460 (RXFP4 ligand)). Competition binding curves for each peptide were performed in triplicate and each experiment was performed independently at least three times. Fluorescent measurements were carried out at excitation of 340 nm and emission of 614 nm on a BMG POLARstar plate reader (BMG Labtech, Melbourne, Australia). Pooled data were presented as mean±S.E.M. of specific binding and are fitted using a one-site binding curve in GraphPad Prism version 8 (GraphPad Inc., San Diego, USA). Statistical analyses were conducted using one-way analysis of variance with uncorrected Fisher's least significant difference (LSD) post-hoc analysis in GraphPad Prism version 9.

3.2—RXFP3 and RXFP4 cAMP Assays

Since RXFP3 is coupled to Gi/o proteins, peptides were tested for their ability to activate RXFP3 as measured via their ability to inhibit forskolin-induced cAMP activity in CHO-K1-RXFP3 or CHO-K1-RXFP4 cells transfected with a pCRE (cAMP Response Element) β-galactosidase reporter plasmid (as described in Belgi, A et al., Structure and function relationship of murine insulin-like peptide 5 (INSL5): free C-terminus is essential for RXFP4 receptor binding and activation. Biochemistry 2011, 50 (39), 8352-61; and Haugaard-Kedstrom, L. M et al., Synthesis and pharmacological characterization of a europium-labelled single-chain antagonist for binding studies of the relaxin-3 receptor RXFP3. Amino Acids 2015, 47 (6), 1267-71). For agonist assays, cells were stimulated with forskolin (5 μM for RXFP3, 1 μM for RXFP4)±increasing concentrations of each peptide for 6 hours. Data were expressed as the % forskolin activity, whereby 100% was defined as forskolin alone and 0% as maximum agonist stimulation (1 μM Analogue 2 for RXFP3 and 1 μM Analogue 13 for RXFP4). Data were analysed and plotted using GraphPad Prism 9 and are expressed as the mean±SEM of the pooled data. Statistical analysis was conducted using one-way ANOVA with Uncorrected Fisher's LSD post-hoc analysis using GraphPad Prism 9.

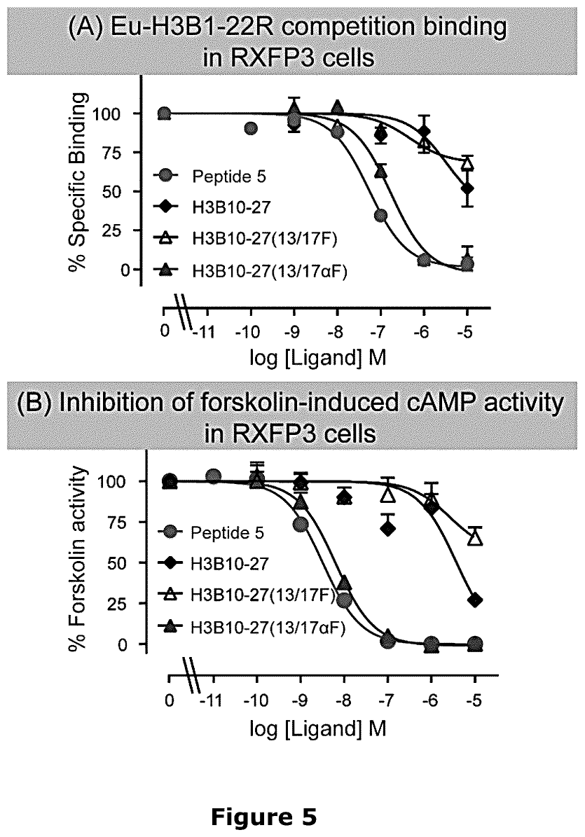

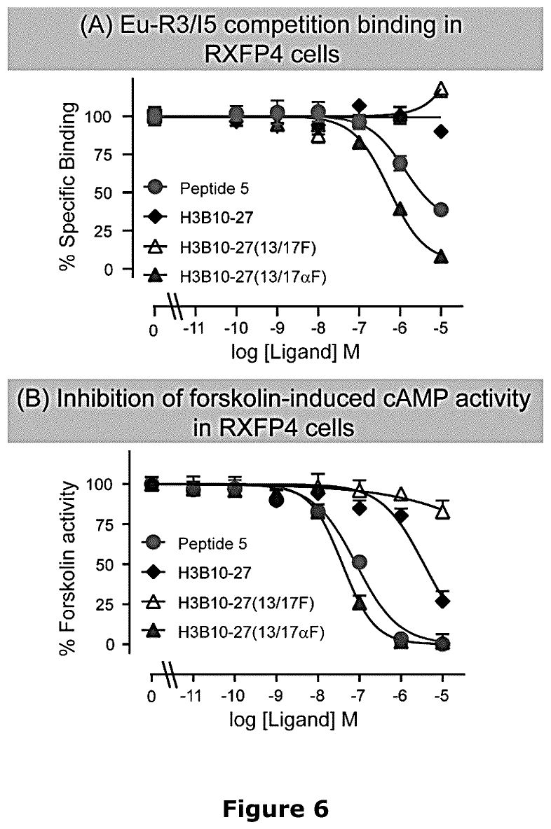

Competition binding and cAMP activity assay results are shown in FIG. 5 (RXFP3) and FIG. 6 (RXFP4). Pooled binding addinity (pKi) and cAMP potency (Pec50) data are shown in Table 2 (RXFP3) and Table 3 (RXFP4) below.

TABLE 2

RXFP3

| Ligand | Eu-H3B1-22R pKi | cAMP pEC50 |
|---|---|---|
| H3 relaxin | 7.64 ± 0.09 (4) | 9.08 ± 0.07 (5)^{#} |
| R3/I5 | 7.77 ± 0.16 (3)^{θ} | 8.89 ± 0.09 (5)\hat{ } |
| Peptide 5 | 7.38 ± 0.03 (3) | 8.48 ± 0.06 (5) |
| H3B10-27(13/17αF) | 6.83 ± 0.04 (3)^{#} | 8.16 ± 0.08 (6)\hat{ } |
| H3B10-27(13/17F) | <5 (3) | <5 (3) |
| H3B10-27 | <5 (3) | <5 (3) |

^{#}p < 0.001, \hat{ }p < 0.01, ^{θ}p < 0.05 vs Peptide 5

TABLE 3

RXFP4

| Ligand | Eu-R3/I5 pKi | cAMP pEC50 |
|---|---|---|
| H3 relaxin | 8.42 ± 0.19 (4)^{#} | 8.94 ± 0.13 (4)^{#} |
| R3/I5 | 8.28 ± 0.07 (7)^{#} | 8.98 ± 0.09 (8)^{#} |
| Peptide 5 | 6.08 ± 0.17 (4) | 6.95 ± 0.10 (4) |
| H3B10-27(13/17αF) | 6.81 ± 0.11 (3)^{θ} | 7.26 ± 0.12 (3)^{θ} |
| H3B10-27(13/17F) | No binding (3) | No activity (4) |
| H3B10-27 | <5 (3) | No activity (3) |

^{#}p < 0.001, ^{θ}p < 0.05 vs Peptide 5

The target peptide, H3B(I-27(13/17αF) (SEQ ID NO 4), exhibited very strong RXFP3 binding affinity. In contrast, the control peptides, H3B10-27(13/17F) (SEQ ID NO 5) and H3B10-27 (SEQ ID NO 6), exhibited very poor binding affinity (FIG. 5A, Table 2).

Consistent with the binding data, H3B10-27(13/17αF) was able to activate RXFP3 with similar potency to Peptide 5, as reflected by inhibition of cAMP production, while the control peptides H3B10-27(13/17F) and H3BT-27 had very low activity (FIG. 5B, Table 2).

The Eu-R3/15 competition binding assay in CHO-KT-RXFP4 cells revealed that, similar to the HC-stapled Peptide 5 (SEQ ID NO 3), H3B10-27(13/17αF), exhibited strong binding affinity for RXFP4, whereas the control peptides H3B10-27(13/17F) and H-3B10-27 had very poor binding affinity (FIG. 6A, Table 3). Consistent with the binding data, H-3B10-27(13/17αF) demonstrated very strong potency at inhibition of cAMP production at RXFP4, similar to Peptide 5, whereas control peptides exhibited no or poor activity (FIG. 6B, Table 3)

### 3.3—B-Arrestin2 Recruitment Assay

It has been previously demonstrated that agonist activation of RXFP3 and RXFP4 results in the recruitment of both β-arrestin1 and β-arrestin2, leading to receptor internalization (Kocan, M. et al., Signalling profiles of H3 relaxin, H2 relaxin and R3(BDelta23-27)R/15 acting at the relaxin family peptide receptor 3 (RXFP3). Br J Pharmacol 2014, 171, 2827-2841; and Ang, S. Y. et al., Signal transduction pathways activated by insulin-like peptide 5 at the relaxin family peptide RXFP4 receptor. Br J Pharmacol 2017, 174, 1077-1089). The inventors assessed if H3B10-27(13/17αF) retained the ability to recruit β-arrestin2 and therefore display full agonist activity at RXFP3 and RXFP4, in comparison to Peptide 5. In this study, the inventors developed a more sensitive β-arrestin2 recruitment assay using Nanoluciferase (NLuc)-tagged RXFP3 (RXFP3-NLuc) and Venus-tagged-β-arrestin2 (Venus-β-Arr2) in a similar manner to that used for RXFP4 (Pustovit, R. et al., A novel antagonist peptide reveals a physiological role of insulin-like peptide 5 in control of colorectal function. ACS Pharmacol Transl Sci 2021, 4, 1665-1674).

The RXFP3 β-arrestin2 recruitment assay was carried out by PCR cloning NLuc onto the C-terminus of human RXFP3 in an identical manner to the RXFP3-rLuc8 construct to create pcDNA3.1-RXFP3-NLuc. The construct was sequenced on both strands to confirm that the NLuc was in frame with RXFP3 and there were no additional PCR mutations. pcDNA3.1-RXFP3-NL was tested in parallel with pcDNA3.1-RXFP3 in cAMP activity assays to determine if the C-terminal nanoluciferase fusion affected agonist activity. Hence constructs were co-transfected in HEK 293T cells with the pCRE-β-galactosidase reporter, as described above, and stimulated with the RXFP3 agonist, R3/I5.

For NanoBRET β-arrestin2 recruitment assays, HEK293T cells were plated onto a six-well plate, and were transfected the following day with β-arrestin2-Venus and either RXFP3-Nluc or RXFP4-Nluc, in the pcDNA3.1/Zeo(+) vector, using Lipofectamine 2000. The next day, cells were washed with DPBS and resuspended in complete media containing 25 mM HEPES but no phenol red, and were seeded into a 96-well CulturPlate (PerkinElmer). The next day, prior to the assay, cells were pre-incubated in phenol red free media with NLuc substrate, 5 μM coelenterazine 400a for RXFP3-nLuc and 5 μM coelenterazine h for RXFP4-NLuc (both from Nanolight, Pinetop, AZ, USA) to establish a baseline, before addition of ligand or vehicle. Emissions were detected simultaneously using a PHERAstar FSX plate reader (BMG Labtech) with 450/80 nm and 535/30 nm filters. Ligand-induced BRET ratio was calculated by subtracting the ratio of 535/30 nm emissions to 450/80 nm emissions for vehicle from the same ratio for ligand-treated wells. For time-courses, ligand-induced BRET ratio was plotted against time, with the last pre-reading before ligand addition displayed as the zero-time point (time of vehicle/ligand addition). Data are representative of 3-5 experiments performed in duplicate and concentration-response curves were fit to data by applying non-linear regression to area under the curve of time-course data, using GraphPad Prism 9.

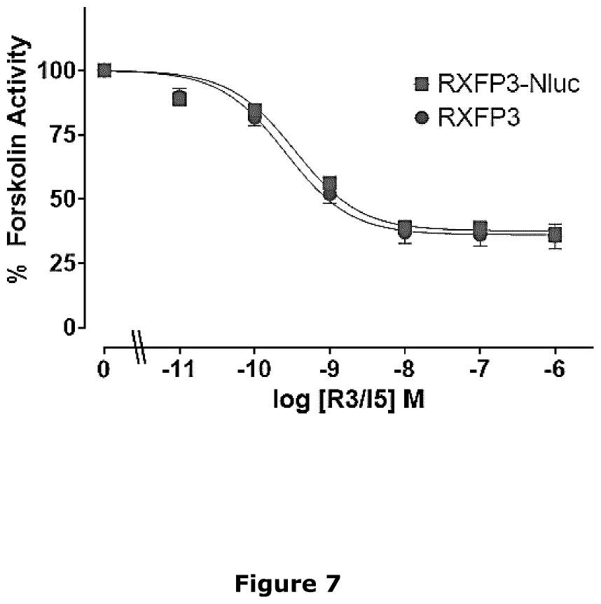

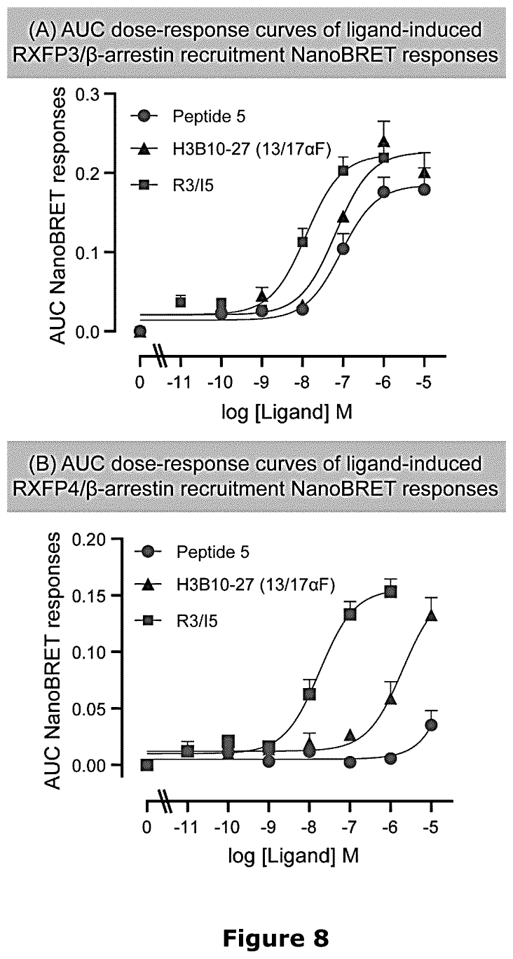

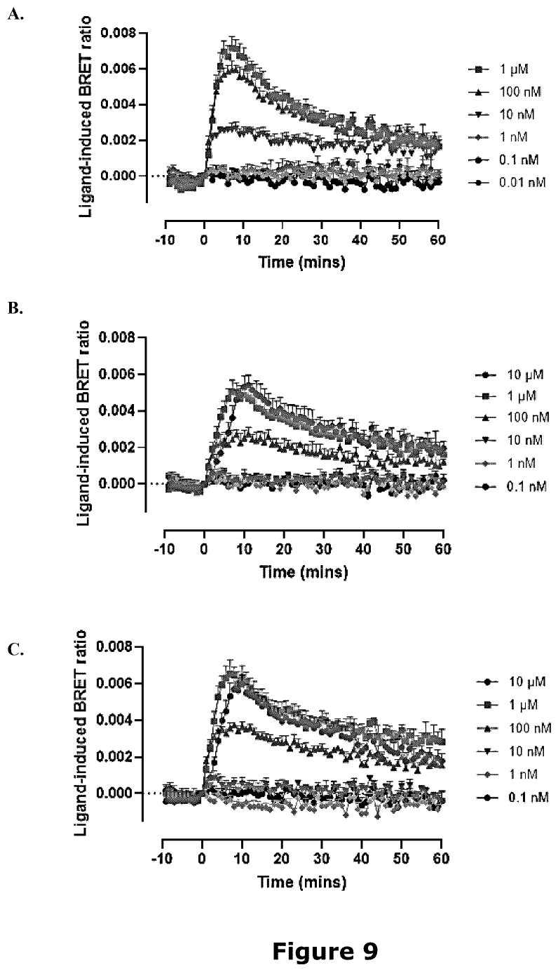

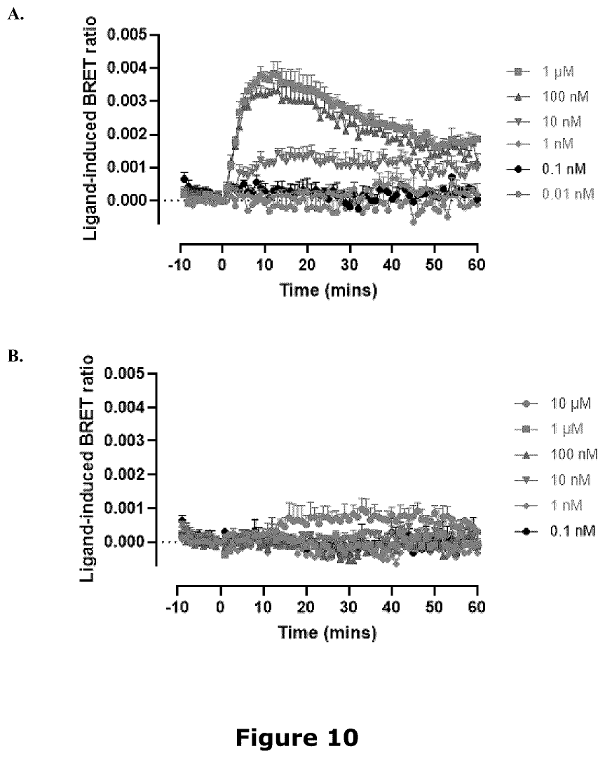

It was first demonstrated that the RXFP3-NLuc construct signalled in response to an RXFP3 agonist, R3/I5, with identical potency and efficacy to responses on untagged RXFP3 (FIG. 7). It was then tested whether R3/15 was able to stimulate a dose-dependent increase in NanoBRET signal in cells co-transfected with Ve-nus-β-Arr2 (FIG. 9A). As anticipated, the BRET signals obtained were more robust than those with rLuc8 and demonstrated a clear ligand-mediated concentration dependence. The responses to Peptide 5 and H3B10-27(13/17αF) were tested in parallel with both peptides displaying dose-dependent recruitment of Venus-β-Arr2 with similar efficacy to R3/15 (FIG. 9B,C). Dose-response curves were plotted based on the area under the curve (AUC) of the entire time course and demonstrated that both Peptide 5 (pEC50=7.19±0.16, p≤0.05) and H3B10-27(13/17αF) (pEC50=7.05±0.17, p≤0.01) showed similar, slightly lower potency compared to R3/15 (pEC50=7.91±0.13) (FIG. 8A and Table 4 below) consistent with their slightly lower affinity and potency in cAMP activity assays. Notably, when Peptide 5 and H3B10-27(13/17αF) were tested in parallel with R3/15 in RXFP4-NLuc/Venus-β-Arr2 recruitment assays, there were marked differences in peptide activity compared to R3/I5 (FIG. 10). Dose-response curves were plotted based on the AUC of the entire time course and demonstrated that Peptide 5 produced only a minimal response at the maximum 10 μM concentration, whereas H3B10-27(13/17αF) was 100-fold less potent than R3/15 (pEC50=5.57±0.26, compared to pEC50=7.56±0.16, p≤0.001) (FIG. 8B and Table 4 below).

TABLE 4

| Ligand | RXFP3 pEC50 | RXFP4 pEC50 |
|---|---|---|
| R3/I5 | 7.91 ± 0.13 (n = 4) | 7.56 ± 0.16 (n = 4) |
| B10-27(13/17αF) | 7.19 ± 0.16^{#} (n = 4) | 5.57 ± 0.26^{###} (n = 3) |
| Peptide 5 | 7.05 ± 0.17^{##} (n = 4) | <5 (n = 3) |
| (B10-27(13/17HC)) | | |

Together, these binding and activity data suggest that the novel analogue H3B10-27(13/17αF) can mimic the biological actions of HC-stapled Peptide 5 in both RXFP3- and RXFP4-expressing cells. However, the lower affinity and potency of both peptides on RXFP4 correlates with a lower potency in β-arrestin2 recruitment.

Analogues that contained α,α-disubstituted amino acids that lack either residues containing phenyl rings, including phenylalanine, or α-methyl groups were unable to mimic the biological properties of RXFP3 agonists, supporting that that both α-methyl groups and phenyl rings of the αF residues are essential to induce π-helical conformation and thus critical to retain their biological function.

### Example 4—Studies of Ex-Vivo Perfused Rat Brainstem

Relaxin-3 is a neuropeptide discovered by the present inventors in 2002 and its endogenous receptor, RXFP3, is abundantly expressed in numerous regions of rodent brain consistent with innervation from nerve fibres expressing relaxin-3 originating from the nucleus incertus, the primary site of relaxin-3 expression (Smith, C. M. et al., Distribution of relaxin-3 and RXFP3 within arousal, stress, affective, and cognitive circuits of mouse brain. J Comp Neurol 2010, 518, 4016-4045). The physiological roles of the relaxin-3/RXFP3 system are emerging and recent data generated by the present inventors' team and others suggest that RXFP3 agonists have potential application for the treatment of neuropsychiatric disorders such as anxiety and depression (Smith, C. M. et al., Relaxin-3/RXFP3 networks: an emerging target for the treatment of depression and other neuropsychiatric diseases? Front Pharmacol 2014, 5, 46; and Ma, S. et al., Distribution, physiology and pharmacology of relaxin-3/RXFP3 systems in brain. Br J Pharmacol 2017, 174, 1034-1048). In order to verify that the novel analogue of the present disclosure retains biological activity in animal models, its activity was examined in two rat models of relaxin-3/RXFP3 function.

The present inventors recently demonstrated that injections of an RXFP3 agonist (A2 analogue, modified form of H3 relaxin with two chains) into the caudal dorsal medulla oblongata modulated respiratory rate and the arterial chemoreceptor reflex in an in situ perfused rat brainstem preparation (Shabanpoor, F. et al., Minimization of human relaxin-3 leading to high-affinity analogues with increased selectivity for relaxin-family peptide 3 receptor (RXFP3) over RXFP1. J Med Chem 2012, 55, 1671-1681). Because the novel agonist of the present disclosure displayed remarkably high stability in serum (FIG. 4), it was examined whether systemic application of the novel agonist, H3B10-27(13/17αF) (SEQ ID NO 4) in the perfusate (blood substitute) could replicate these results.

Juvenile Sprague-Dawley rats (postnatal day 18-23, weighing 40-60 g) were used. Rats were maintained on a 12 h light/dark cycle with food and water available ad libitum. All experiments followed protocols approved by The Florey Institute of Neuroscience and Mental Health Animal Ethics Committee and performed in accordance with the NHMRC Code of Practice for the Use of Animals for Scientific Purposes. Recently published methods were followed (Furuya, W. I. et al., Relaxin-3 receptor (RXFP3) activation in the nucleus of the solitary tract modulates respiratory rate and the arterial chemoreceptor reflex in rat. Respir Physiol Neurobiol 2020, 271:103310).

Briefly, rats were deeply anaesthetized via inhalation of isoflurane (Zoetis, Sydney, Australia) until complete loss of the hindpaw withdrawal reflex in response to noxious pinch. They were bisected below the diaphragm, and immediately immersed in ice-cold Ringer's solution, skinned, eviscerated and decerebrated at the pre-collicular level. The right thoracic phrenic nerve was isolated and cut distally at the level of its insertion into the diaphragm muscle to record the efferent activity. The heart and lungs were removed and the preparation was transferred to a recording chamber. A double-lumen catheter was inserted into the descending aorta for retrograde perfusion of carbogenated Ringer's solution, heated to 31° C. using a peristaltic pump (Watson & Marlow 505 S, Falmouth, UK). The second lumen of the catheter was used to monitor aortic perfusion pressure, which was maintained in the range 40-70 mmHg by adjusting the flow at 18-22 mL/min. The perfusate was an isosmotic Ringer's solution containing, in mM: NaCl, 125.00; NaHCO₃, 24.00; KCl, 3.00; CaCl₂), 2.50; MgSO₄, 1.25; KH₂PO₄, 1.25; and glucose, 10.00) containing an oncotic agent (0.45% sucrose), bubbled with carbogen (95% 02 and 5% CO₂), pH 7.4 after carbogenation, and filtered using a nylon screen (pore size 100 μm; Millipore, Tullagreen, Ireland). After respiratory-related movements commenced, a neuromuscular blocker (vecuronium bromide, 300 μg/200 ml perfusate, Mylan, Brisbane, Australia) was added to the perfusate to mechanically stabilize the preparation. The eupneic respiratory pattern was obtained by stimulating the peripheral chemoreflex with a bolus injection of NaCN (100 μL, 0.1% w/v).

The phrenic nerve activity (PNA) was recorded from the cut proximal nerve end using a suction electrode. The signal was amplified (differential amplifier DP-311, Warner instruments, Hamden, USA), band-pass filtered from 100 Hz to 10 kHz, digitized (PowerLab/16SP ADInstruments, Sydney Australia) and then viewed and recorded using LabChart (version 7.0, ADInstruments).

After the preparation stabilized, a minimum of 10 min baseline activity was recorded. Then, the peripheral chemoreflex was evoked at least 2 times by a bolus injection of NaCN (100 μL, 0.1% w/v) under control conditions. At 5 min after the last control chemoreflex stimulus, H3B10-27(13/17αF) was added to the perfusate (2 μM final concentration). Following an 8-10 min interval, the chemoreflex stimuli were repeated, as described.

Analyses of the PNA were performed using Spike2 software version 7.12 (Cambridge Electronic Design, Cambridge, UK). The phrenic nerve burst frequency was analysed over a 1 min period during the baseline recording and after the systemic application of H3B10-27(13/17αF), immediately before the second chemoreflex stimulus. The respiratory response to the chemoreflex was analysed by counting the number of phrenic nerve bursts during the tachypneic response. Results are expressed as the mean±SEM and analysed by paired t-test, using SigmaPlot statistics software 11.0. The level of significance used was p≤0.05.

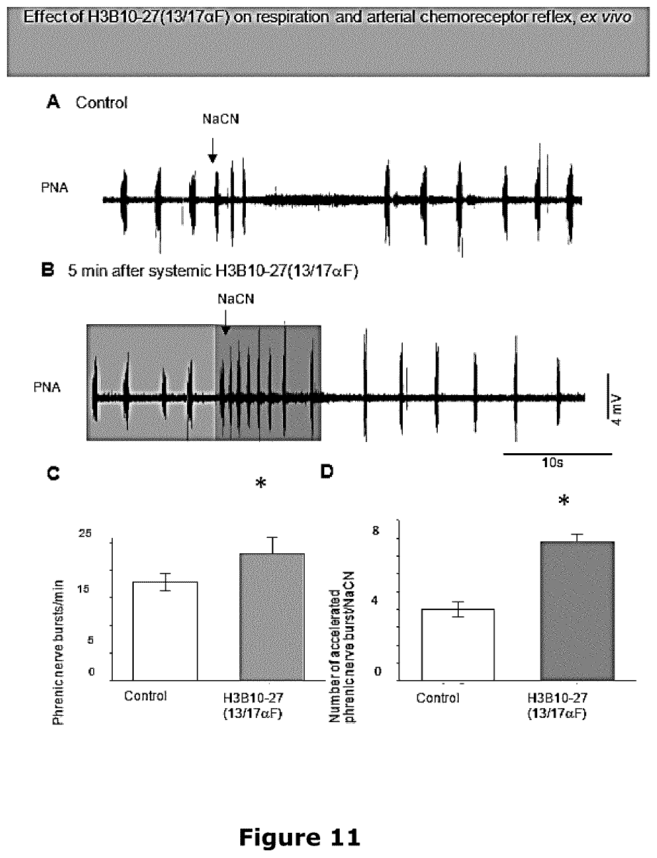

Similar to centrally injected A2 (Furuya, W. I. et al., Relaxin-3 receptor (RXFP3) activation in the nucleus of the solitary tract modulates respiratory rate and the arterial chemoreceptor reflex in rat. Respir Physiol Neurobiol 2020, 271:103310), systemic application of H3B10-27(13/17αF) significantly increased phrenic nerve activity (PNA) 17.8±1.6 to 23.0±2.9 PNA-bursts/min (FIG. 11; +27.7±5.4%, p=0.014, n=6). In addition, systemic application of H3B10-27(13/17αF) also significantly enhanced the arterial chemoreceptor reflex-mediated tachypnea from 4.0±0.4 tachypneic PNA bursts to 7.8±0.5 PN bursts (FIG. 11; p≤0.001, n=6). This ex vivo data, together with in vitro data (FIGS. 5, 6 and 8) confirmed that the new single-chain analogue mimics the pharmacological action of the RXFP3 agonists, Peptide 5 (SEQ ID NO 3) and A2.

### Example 5—In Vivo Feeding Studies in Rats (RXFP3 Agonist)

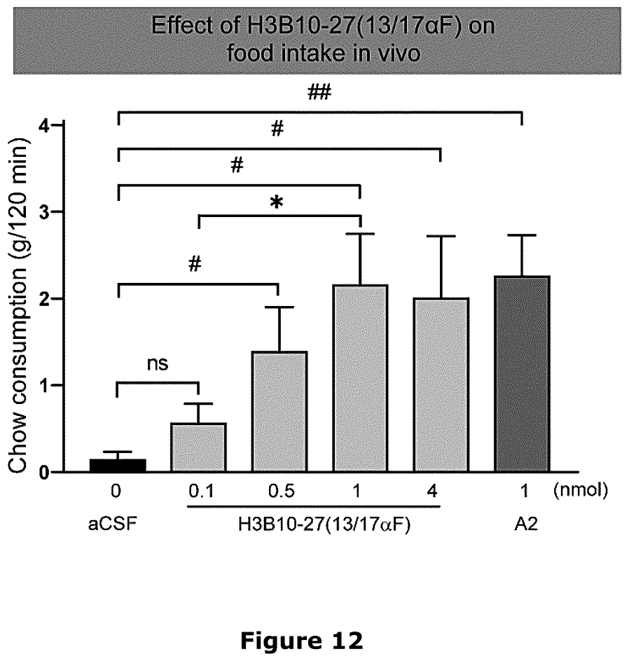

Central (icv) administration of RXFP3 agonists in rats is known to increase food intake (Hojo, K. et al., Development of a single-chain peptide agonist of the relaxin-3 receptor using hydrocarbon stapling. J Med Chem 2016, 59, 7445-7456; and Shabanpoor, F. et al., Minimization of human relaxin-3 leading to high-affinity analogues with increased selectivity for relaxin-family peptide 3 receptor (RXFP3) over RXFP1. J Med Chem 2012, 55, 1671-1681). Therefore, the peptide of the present disclosure, H3B10-27(13/17αF) (SEQ ID NO 4) was evaluated in adult, male rats, and its effect on food intake compared with the RXFP3 agonist, A2 (FIG. 12).

Male, adult Sprague-Dawley rats (n=29, weighting 250-300 g; Janvier Labs, Le Genest-Saint-Isle, France) were used. Rats were housed under ambient conditions (21° C.) and maintained on a 12 h light/dark cycle (lights on 08:00-20:00), with access to food (laboratory chow) and water ad libitum. Rats were acclimatized to the animal facility for at least 1 week before further treatment. Experiments were conducted with the approval of the Committee of Ethics in Animal Welfare of the Universitat Jaume I and registered in the Conselleria de Agricultura of the Generalitat Valenciana (Spain). All efforts were made to minimize the number of animals used and special attention was taken to minimize any suffering or discomfort.

Rats were anesthetized with isoflurane (Isoflutek®, Laboratorios Karizoo, Barcelona, Spain) for the implantation of cannula into the lateral ventricle. An initial anesthesia was induced with 4% isoflurane in oxygen, 2 L/min, and under deep anesthesia, rats were placed in a stereotaxic frame (David Kopf Instruments, Tujunga, CA, USA) and maintained with 2-3% isoflurane in oxygen, 200 mL/min. The skull was positioned and a stainless-steel guide cannula (22 gauge) was implanted with the cannula tip aimed at the lateral ventricle (coordinates: anteroposterior, 0.2 mm; mediolateral, −1.5 mm; dorsolateral, −3.7 mm). The cannula was attached to the skull using surgical screws and dental cement. When recovered, rats were placed individually in clean cages. Meloxicam (Metacam® Boehringer Ingelheim Vetmedica GmbH, Rhein, Germany) was subcutaneously injected at a dosage of 0.5 mg in 0.1 mL (for 2 days), to provide acute and ongoing post-operative analgesia. Rats were single-housed and allowed to recover for 7 days, during which they were handled and weighed daily to habituate them to the experimenter. A dummy stylet of stainless steel wire (30 gauge) was inserted into each cannula to maintain patency.

Lateral cerebral ventricle infusions were made using a 30-gauge stainless steel hypodermic tubing injector (Plastics One, East Virginia, USA) connected to a 10-μL Hamilton microsyringe (Hamilton Instruments, Reno, NV, USA) by polyethylene tubing (0.80 mm outer and 0.40 mm internal diameter; Plastics One). On post-surgery day 3, correct positioning of the cannula was verified in each rat by injecting 5 μL of a 2 ng/μL solution of angiotensin II (Hello Bio HB3488, Dunshlaughlin, Ireland) in artificial cerebrospinal fluid [aCSF; made from stock of 10×aCSF (1470 mM NaCl, 40 mM KCl, 8.5 mM MgCl₂, and 23 mM CaCl₂))], and observing if this produced a dipsogenic response, defined as repeated drinking episodes of ≥5 s that commenced within 1 min of angiotensin II administration.

Each rat received 3 icv infusions on days 7, 12 and 15 post-surgery. Rats were handled 3 days before the infusions started and left in the infusion room on the previous day. Infusions were performed 2 h after lights on. The injection volume was 5 μL in all cases and contained either aCSF (n=8), 0.1 nmol H3B10-27(13/17αF) (n=10), 0.5 nmol H3B10-27(13/17αF) (n=10), 1 nmol H3B10-27(13/17αF) (n=14), 4 nmol H3B10-27(13/17αF) (n=12) or 1 nmol A2 (n=15) in 6 groups of injections in total. A pre-weighed water bottle was also placed in its usual compartment. Food and water were weighed at intervals of 15, 30, 60 and 120 min after the infusion. Thereafter, the cages were returned to the home room.

At the end of the experiment, rats were anesthetized with an overdose of 125 mg/kg sodium pentobarbital (Dolethal, Vetaquinol, Madrid, Spain) to check cannula placement. Rats were transcardially-perfused with saline, followed by 4% paraformaldehyde in 0.1 M phosphate buffer. Brains were removed from the skull, and kept in the same fixative overnight at 4° C., then transferred to 30% sucrose in 0.01M phosphate buffer saline for cryoprotection. Brains were coronally sectioned with a sliding microtome and examined.

Food and water intake data were analyzed using GraphPad Prism 5. Results are expressed as mean±SEM. A D'Agostino and Pearson normality test was applied. Under normality conditions an unpaired t test was applied to compare groups. When normality was not achieved, a non-parametric Mann-Whitney U test was applied to compare between medians. The level of significance used was α=0.05.

An icv infusion of vehicle (aCSF) did not produce feeding or any marked ingestion of chow (FIG. 12). Infusion of 0.1 nmol of H3B10-27(13/17αF) (n=10), produced a trend towards increased food intake, relative to aCSF (n=8), but this was not significant (p=0.077). However, infusion of higher amounts of H3B10-27(13/17αF) produced significant, dose-dependent increases in food intake relative to aCSF [i.e., differences were observed between aCSF and 0.5 nmol B10-27(13/17αF) (n=10; p=0.019, Mann-Whitney U test), aCSF and 1 nmol B10-27 (n=14; p=0.019, Mann-Whitney U test); aCSF and 4 nmol B10-27(13/17αF; p=0.014, Mann-Whit-ney U test)]; or aCSF and A2 (n=15) (p=0.003, Mann-Whitney U test). In a further comparison, a significant difference was observed between the 0.1 nmol and 1 nmol B10-27(13/17αF) (p=0.042; t=2.162, df=22; D'Agostino and Pearson normality test and unpaired t-test) (FIG. 12). The maximum effect observed was comparable to that of an infusion of the RXFP3 agonist, A2 (1 nmol A2 in 5 μl).

### Example 6—Preparation of Stapled Insulin-Like Peptide 5 (INSL5)

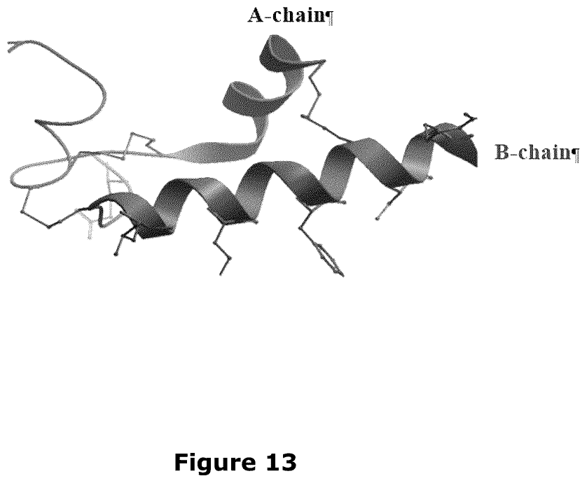

INSL5 belong to the insulin superfamily and comprises, in its native form, an A- and B-chain linked by three disulphide bonds (FIG. 13), according to Formula I below.

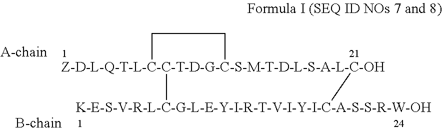

The synthesis and purification of both the A- and B-chain of INSL5 is difficult, due to their hydrophobic and aggregative nature. Existing multi-step synthesis and purification of INSL5 is time consuming and low-yielding. It is also difficult to modify the two-chain structure of INSL5 for improvement of its pharmacokinetic properties.

The smallest two-chain analogue of INSL5 acting as a potent agonist on the receptor RXFP4 is INSL5-A13:B7-24 (Formula II). Most of the key residues for receptor binding and activation are located on the B-chain.

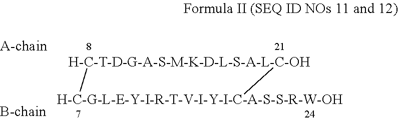

The present inventors have found that substitution of GlyGly (GG) at positions B20 and B21 give a significant improvement in the binding and affinity (Formula III) (INSL5-A13:B7-24_G20/21). The potency of the INSL5-A13: B7-24_G20/21 is ~10 times more than two chains simplified agonist, exceeding even the native INSL5, resulting in the development of a superagonist.

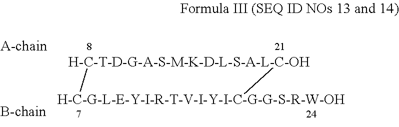

The present inventors then sought to develop B-chain-specific biologically active analogues, according to Formula IV:

Formula IV
(SEQ ID NO. 15)
7 24
S-G-L-E-Y-I-R-T-V-I-Y-I-S-G-G-S-R-W-OH

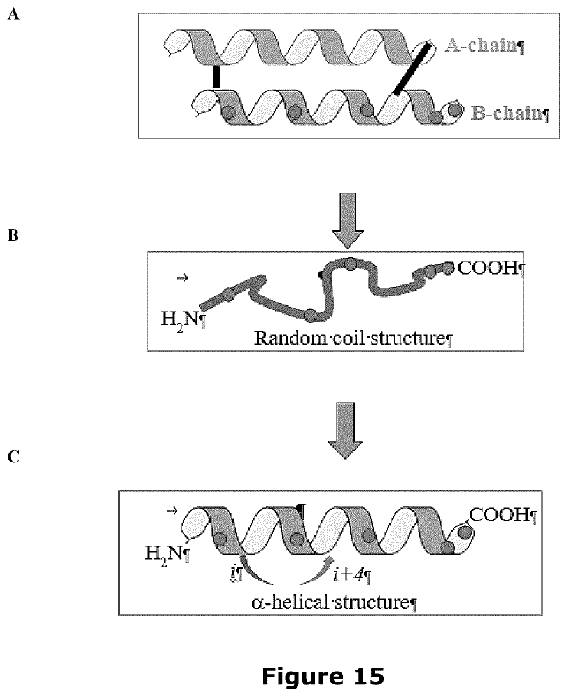

Without A-chain support, this isolated B-chain of INSL5 is unstructured and thus, inactive. It has a random coil structure (FIG. 15B). Stapling was employed to enhance the tendency of the linear B-chain to adopt the right conformation for receptor binding even in the absence of A-chain support. Stapling peptide modifications were made at positions 10 and 14 since these two positions have a minor contribution to binding and activation (FIG. 15C).

The peptide was stapled with two αF residues as described herein (formula V, SEQ ID NO. 16). The acetylated analogue thereof (SEQ ID NO. 17) was also prepared in analogous fashion. Peptides stapled with alternative stapling mechanisms were also prepared for comparison, as shown below. In particular, the following stapled peptides were prepared: a hydrocarbon stapled analogue (formula VI (SEQ ID NO. 18)); a lactam bridge stapled analogue (formula VII (SEQ ID NO. 19)); a thioacetal stapled analogue (formula VIII (SEQ ID NO. 20)); and a disulfide bridge stapled analogue (formula IX (SEQ ID NO. 21)). Yields of each product are given in Table 5 in Example 8 below. The residue numberings of SEQ ID NOS. 16 to 21 and 26 are inherited from those of the B chain of native INSL5 (SEQ ID NO. 8).

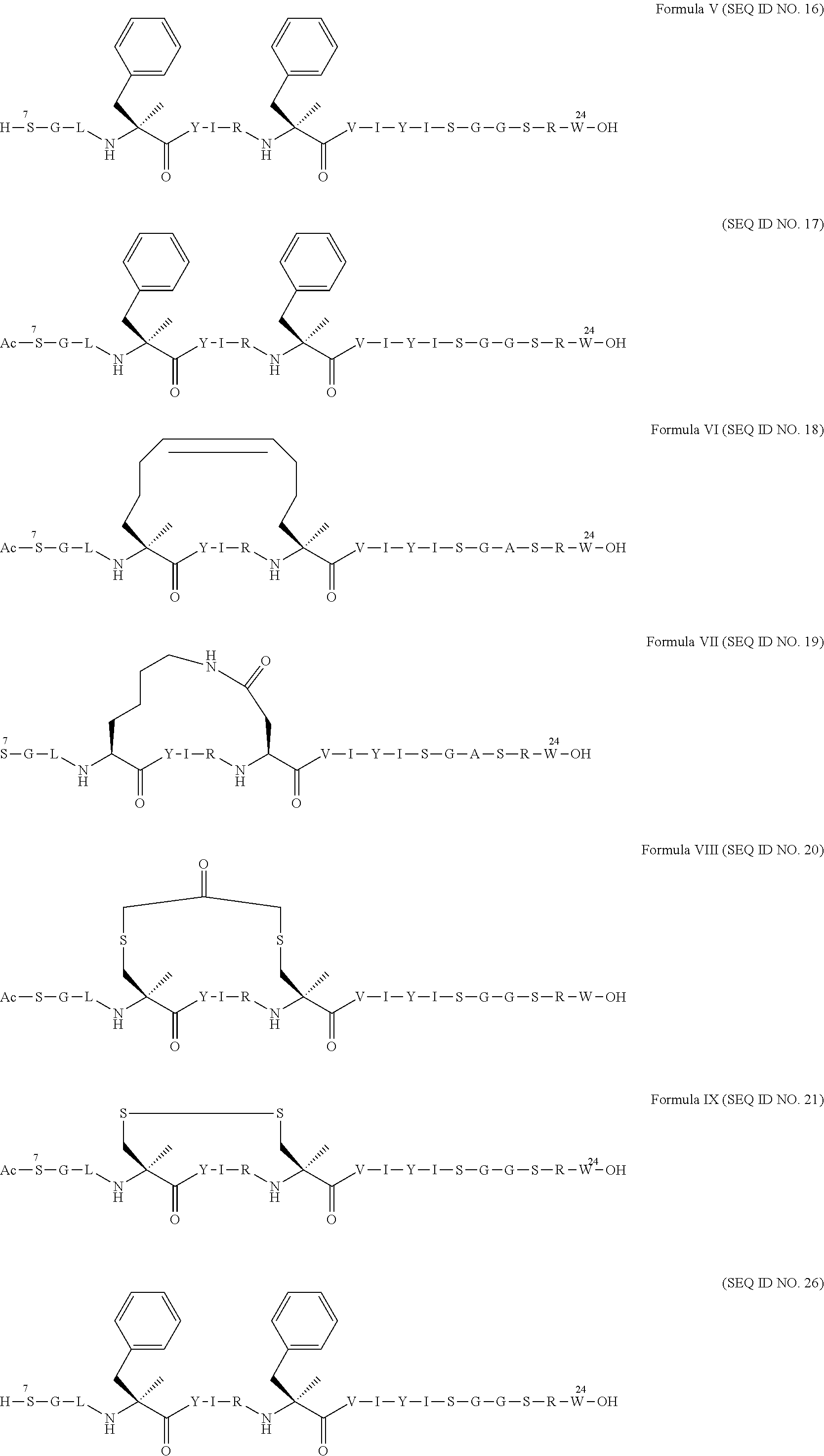

The π-π-stapled INSL5 analogue of Formula V, along with those corresponding to SEQ ID NO. 17 and SEQ ID NO. 26 were synthesized using the standard Fmoc-SPPS synthesis method utilizing two αF (method is as same as that used for H3 relaxin π-π stapled analogues described in Example 1 above, with an acetylation step for SEQ ID NO. 17), and the peptides were ready after TFA cleavage and HPLC purification. The yield of Formula V was higher (>40%) compared with other stapled peptides, as discussed below.

Hydrocarbon-stapled INSL5 analogues (Formula VI): The protected peptides containing S5 ((S)-2-(4-pentenyl)alanine) were subjected to on-resin RCM with Grubbs catalyst. Briefly, 5% 0.4 M LiCl in DMF and 0.2 eq. 2nd generation Grubb's catalyst in DCM was added to 0.02 mmol Fmoc-protected on-resin peptide. All mixtures used were degassed before and after addition to the resin. The reaction mixture was shaken in the dark at room temperature for overnight. Standard cleavage and deprotection followed by preparative RP-HPLC were carried out as described above. The yield of this peptide was >10%.

Lactam-stapled INSL5 analogues (Formula VII): After assembly of the peptide containing Lys10(Mtt) and Asp14(O-2-PhiPr), side chain protection of Lys and Asp were removed on-resin by TFA/TIPS in DCM (2% and 5%, 5 ml) for 30 min, and washed by DCM, 5% DIEA in DMF and DMF. Lactam bond cyclisation was performed by treating with PyClock (2 eq., 28 mg) and DIEA (>20 eq., 100 μl) in DMF (3 ml) overnight. Standard cleavage and deprotection followed by preparative RP-HPLC were carried out as described above. The yield of this peptide was 4%.

Thioacetal-stapled INSL5 analogues (Formula VIII): Purified disulphide bond stapled analogues were dissolved in 30% acetonitrile in H₂O. 1.1 eq. TCEP was added to analogues containing a minimal amount of AB buffer dissolved in 1M ammonium bicarbonate, and the reaction was stirred for 1 h at room temperature. 1.5 e.q. DCA solution (10 mg DCA/1 mL ethanol) was then added to the reaction and further stirred for 2 h at room temperature. The thioether stapling reaction was quenched using 4% TFA and the sample was s diluted with buffer A before purification using RP-HPLC. The thioether stapling process was stopped with 4% TFA, and the samples were diluted with buffer A before RP-HPLC purification. The yield of this peptide was >10%.

Disulfide-stapled INSL5 analogues (Formula IX): After synthesis of the peptide containing α-methyl-Cys10(Trt) and α-methyl-Cys14(Trt), standard cleavage and purification were carried out. Then the linear peptide was treated with 2,2′-dipyridyl sulphide with DIEA in 20% acetonitrile solution at pH 8.5 and 40° C. for 1 hr to form the intramolecular disulphide bond which was purified by preparative RP-HPLC. The yield of this peptide was 20%.

### Example 7—In Vitro Stability of INSL5 Analogues

Serum stability of INSL5 analogues was assessed as described for H3 relaxin in Example 2 above, with the exception of the use of 25% serum (in contrast to 100% serum used for H3 relaxin).

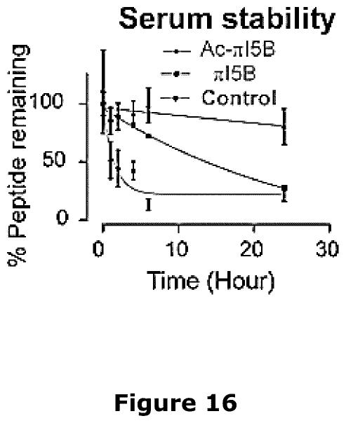

Results are shown in FIG. 16 (control=Linear B chain (SEQ ID NO. 15; Formula IV), πI5B=αF-stapled B-chain analogue (SEQ ID NO. 16; Formula V), AC-πI5B=N-terminal acetylated αF-stapled B-chain analogue (SEQ ID NO. 17).

### Example 8—Cell-Based Assays for Binding and Potency

Binding and cAMP assays for INSL5 analogues were carried out as described above for H3 relaxin (Example 3). An additional analogue of INSL5 was utilised as a control, INSL5-A13, having an A chain (SEQ ID NO: 9) and a B chain (SEQ ID NO: 10) with a first disulfide bond between Cys 1 of the A chain and Cys 7 of the B chain, and a second disulfide bond between Cys 14 of the A chain and Cys 19 of the B chain:

A chain
(SEQ ID NO: 9)
CTDGASMKDLSALC
B chain
(SEQ ID NO: 10)
KESVRLCGLEYIRTVIYICASSRW

Furthermore, a SmBiT-R3/15 competition binding assay was performed. All peptides were examined for their binding affinity to RXFP4 utilizing a competition NanoLuc® Binary Technology (NanoBiT™) binding assay. Briefly, HEK-LgBiT-RXFP4 crude membrane fractions (25 μg protein) were incubated with 2 nM SmBiT-R3/15 and increasing concentrations of competing peptides in binding buffer (0.5% BSA, 20 mM HEPES pH 7.5, 1.5 mM CaCl₂), 50 mM NaCl) in a total of 100 μL in 96-well Optiplates (PerkinElmer, Waltham MA, USA). After 1 hour of shaking at room temperature, 1.25 μM final concentration of coelenterazine 400a was added, and luminescence measurements were recorded using a MicroBeta Microplate Counter (PerkinElmer, Waltham MA, USA). Each concentration-response curve was tested in triplicate, and each experiment was repeated at least three times with data being expressed as the mean±standard error of the mean (S.E.M) % specific.

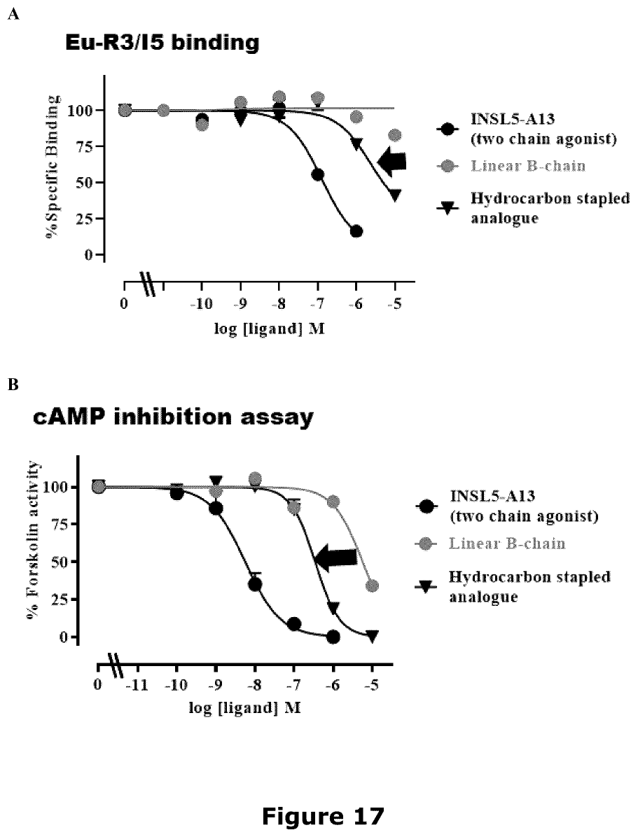

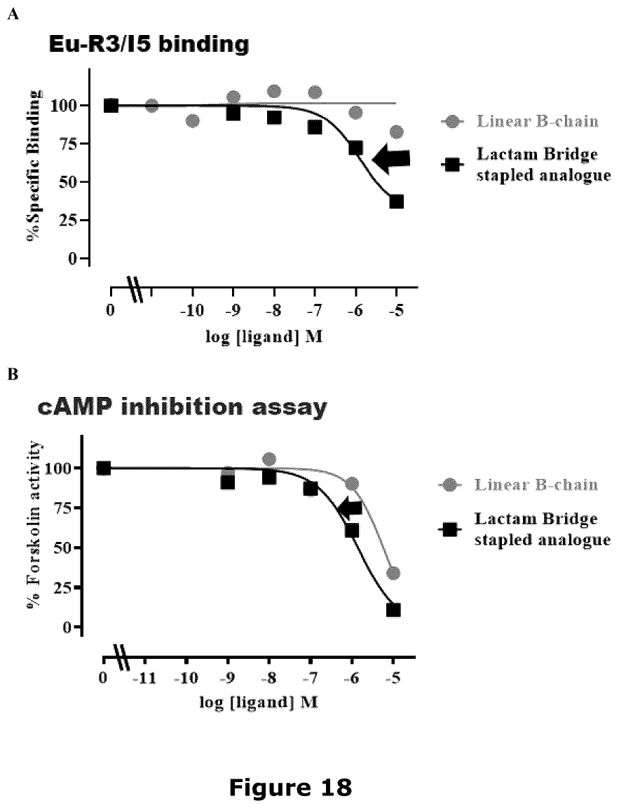

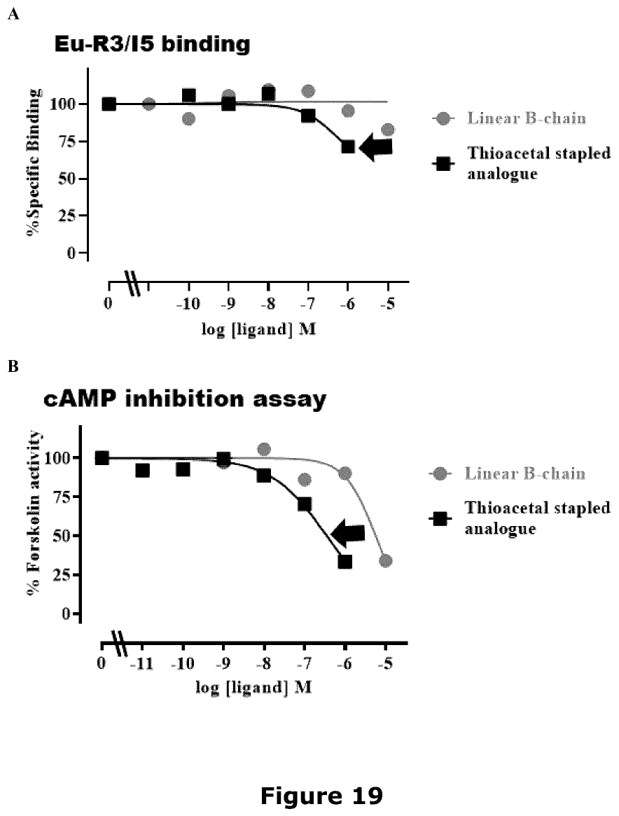

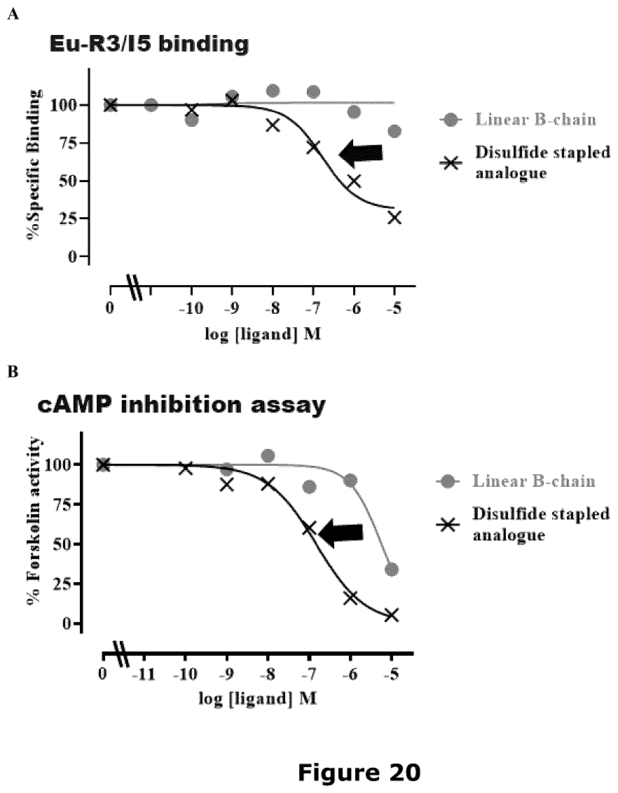

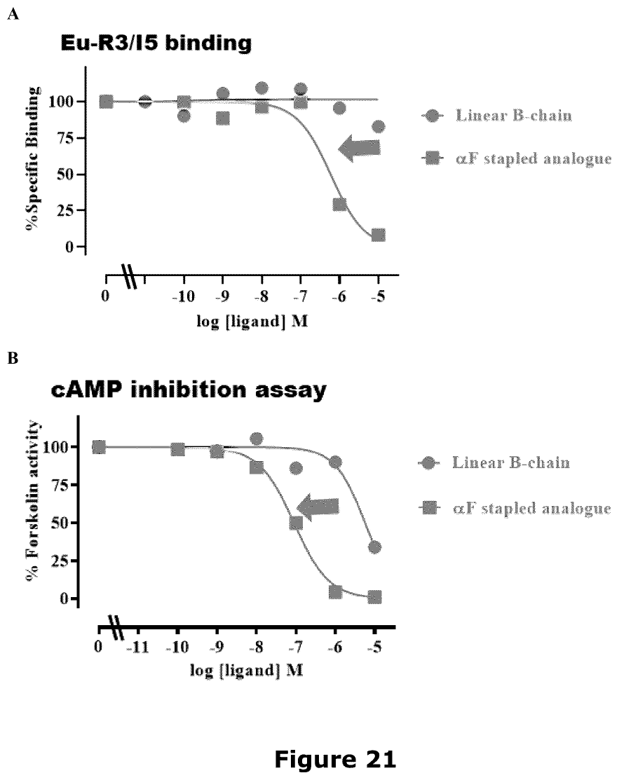

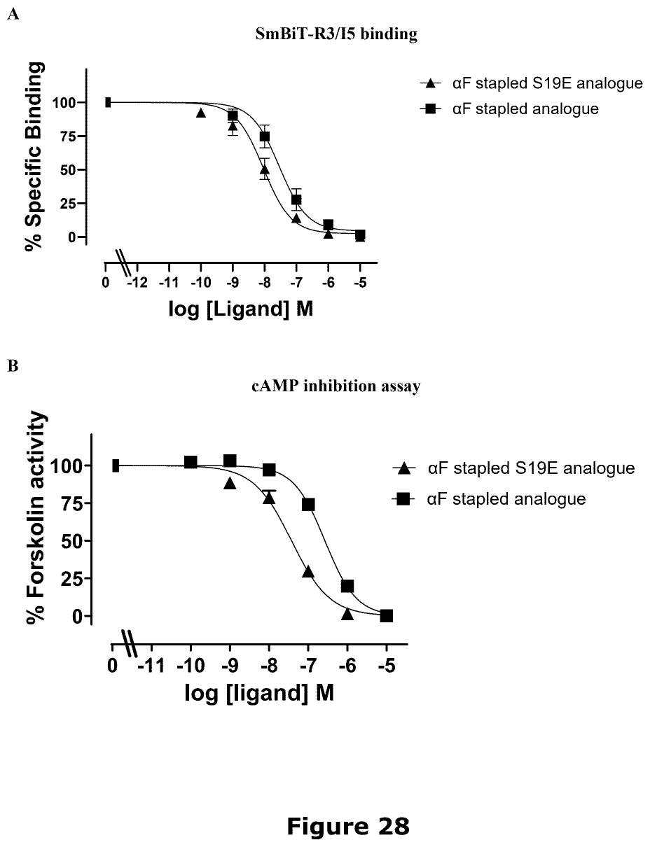

Results are shown in Table 5 below and in FIGS. 17-21 and 28.

TABLE 5

| | | SmBiT R3/I5 | cAMP | |
|---|---|---|---|---|
| Peptide | Eu-R3/I5 pKi | pKi | pEC50 | Yield |
| Native INSL5 (SEQ ID NOs 7 | 8.43 ± 0.05(3) | 8.47 ± 0.14(4) | 8.34 ± 0.22(3) | 0.8% |
| and 8) | | | | |
| INSL5-A13 (SEQ ID NOs 9 | 7.46 ± 0.06 (7) | 8.11 ± 0.05(4) | 8.10 ± 0.10 (4) | 14% |
| and 10) | | | | |
| INSL5-A13: B7-24 (SEQ ID | ND | 8.06 ± 0.11(3) | 8.08 ± 0.11(3) | 13.1% |
| NOs 11 and 12) | | | | |
| INSL5-A13: B7-24_G20/21 | ND | 8.64 ± 0.16(3) | 8.93 ± 0.11(3) | 14% |
| (SEQ ID NOs 13 and 14) | | | | |
| Linear B-chain (SEQ ID NO | No binding (3) | ND | <5 (4) | 58.33% |
| 15) | | | | |
| Hydrocarbon stapled analogue | ND | 6.81 ± 0.14(3) | 6.18 ± 0.15 (3) | 11.05% |
| (SEQ ID NO 18) | | | | |
| Lactam Bridge stapled | ND | 6.45 ± 0.17(3) | 5.93 ± 0.05 (4) | 3.87% |
| analogue (SEQ ID NO 19) | | | | |
| Thioacetal stapled analogue | <5 (3) | ND | 6.29 ± 0.03 (3) | |
| (SEQ ID NO 20) | | | | |
| Disulfide bridge stapled | 7.33 ± 0.11 (3) | ND | 6.84 ± 0.07 (3) | 20.9% |
| analogue (SEQ ID NO 21) | | | | |
| aF stapled analogue (SEQ ID | 6.80 ± 0.14 (3) | 7.51 ± 0.23(3) | 7.06 ± 0.10 (3) | 40.9% |
| NO 16) | | | | |
| πI5B7-24-GG-S19E | ND | 8.13 ± 0.19(3) | 7.39 ± 0.06(3) | 41.6% |
| (SEQ ID NO 26) | | | | |

### Example 9—Constipation Treatment Using INSL5 Mimetics

Male C57B16 mice (20-30 g; ARC), between the ages of 3 to 5 months, were used for all studies. All experiments were conducted in accordance with the National Health and Medical Research Council (NHMRC) guidelines for the care and use of animals and with approval from the Florey Institute of Neuroscience and Mental Health Animal Ethics Committee (#18-128 FINMH and #18-081 FINMH). All animals were group housed (2-6 per cage) and maintained in a humidity-controlled room at 22° C. under a 12 hours light/dark cycle with access to food and water ad libitum.

Loperamide (Sigma-Aldrich) was prepared in 1% Tween-80 (SigmaAldrich) in distilled water and was administered subcutaneously (s.c.) at the back of the neck to induce constipation. Mice were injected with either vehicle (1% Tween-80/water) or loperamide (0.3, 1, 3 mg/kg). INSL5-A13 (20 μg/kg) (SEQ ID NOs 9 and 10) and single-chain αF stapled analogue (200 μg/kg) (SEQ ID NO 16) were dissolved in distilled water and injected intraperitoneally (i.p.) 10 minutes after loperamide was administered (1 mg/kg, s.c.). At 20 minutes post peptide injection, mice were lightly anesthetized with 2% (v/v) isoflurane in O2, administered at 1 L/min for a maximum of 15 seconds following induction with 5% isoflurane in O2 at 1 L/min. A 3-mm round bead was inserted 2 cm into the distal colon using a flexible plastic rod. After bead insertion, mice were placed in individual cages. The time taken from bead insertion to bead expulsion was recorded. A higher mean expulsion time indicated stronger inhibition of colonic propulsion. The maximum time allowed for bead expulsion before manual removal was 30 minutes. If bead expulsion took longer than 30 minutes, the bead was manually removed by gently massaging the bead down the colon until it was expelled. The time was scored as 30 minutes. To ensure there was no desensitization following repeated exposure to loperamide, mice were treated with loperamide at the end of the experimental period to ensure a constipation phenotype could still be induced. All experiments were performed in the afternoon (between 13:00 and 15:00 pm) and all data were used for analysis.

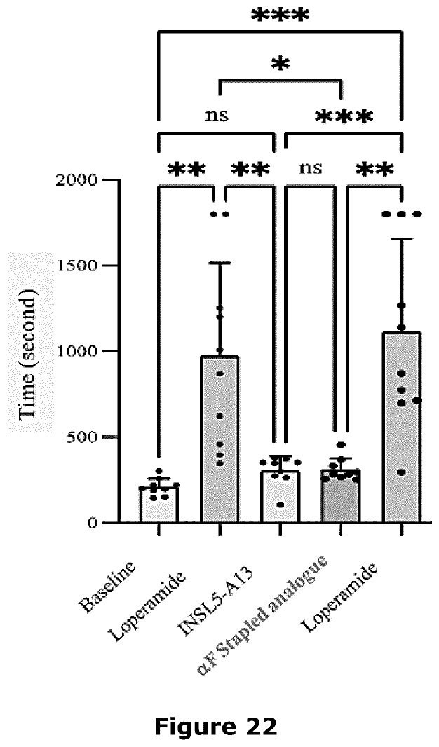

INSL5-A13 (two-chain peptide comprising two-disulfides) (SEQ ID NOs 9 and 10) is a full agonist at RXFP4, and is effective in accelerating colorectal propulsion. The single-chain αF stapled analogue prepared above showed a similar effect to the two-chain agonist (FIG. 22; n=10, male).

### Example 10—RXFP3 Agonist Reduces Pain

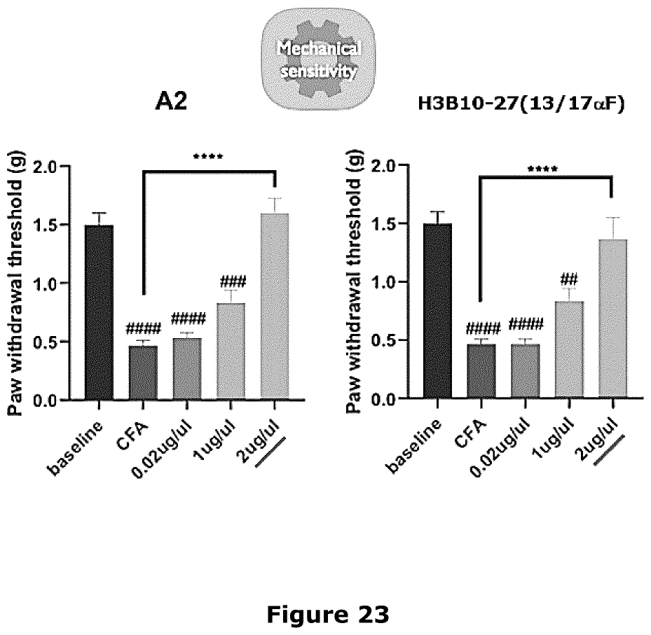

The relaxin 3/RXFP3 system is known to regulate neurological functions (e.g. stress, anxiety) whose dysfunction is often associated with painful pathologies. Furthermore, relaxin-3 neurons project to many forebrain areas involved in pain processing and underlying the sensory or the emotional component of pain. Therefore, the peptide of the present disclosure, H3B10-27(13/17αF) (SEQ ID NO 4) was evaluated in adult mice, and its effect on pain compared with the RXFP3 agonist, A2. The results are depicted in FIG. 23.

Pilot studies were conducted in a mouse model of inflammatory persistent pain obtained by intraplantar injection of Complete Freund's Adjuvant (CFA). RXFP3 activation with intra-cerebroventricular injection of the double-chain (A2) or single chain (π-π stapled, H3B10-27(13/17αF)) agonists induced dose-dependent antinociceptive effects. The effect is maximal at 30 minutes after the injection and vanished after 1 hour. The same result has been obtained in a rat CFA model with a similar time course of the antinociceptive effect. By using local injections, it was also demonstrated that A2 and H3B10-27(13/17αF) induced mechanical (von Frey test) and thermal (plantar test) analgesia when injected in the Anterior Cingulate Cortex (ACC) or the Baso-Lateral Amygdala (BLA), contralateral to the CFA injected paw. The antinociceptive effects were fully abolished by co-injection of the R3(B1-22) RXFP3 antagonist.

### Example 11—RXPF3 Antagonist Inhibits Food Consumption

Central administration of RXFP3 agonists in rats is known to increase feeding behaviours (Hojo, K. et al., Development of a single-chain peptide agonist of the relaxin-3 receptor using hydrocarbon stapling. J Med Chem 2016, 59, 7445-7456; and Shabanpoor, F. et al., Minimization of human relaxin-3 leading to high-affinity analogues with increased selectivity for relaxin-family peptide 3 receptor (RXFP3) over RXFP1. J Med Chem 2012, 55, 1671-1681) and that of antagonists inhibits food consumption and addictive behaviours. These findings suggest that RXFP3 is a potential target for pharmacological control of eating and addictive disorders. Accordingly, an RXFP3 antagonist (Formula X, SEQ ID NO 22) was developed, incorporating two α-methyl phenylalanine at the 13 and 17 positions of H3B1-22R (truncating to remove nine residues from the N-terminus that are unimportant for binding). Three controls were also synthesized (Formulae XI to XIII), including Formula XI (SEQ ID NO 23), a known single chain antagonist of RXFP3 (Haugaard-Kedstrom et al., Design, Synthesis, and Characterization of a Single-Chain Peptide Antagonist for the Relaxin-3 Receptor RXFP3. J Am Chem Soc 2011, 133 (13), 4965-4974), a truncated form thereof Formula XII (SEQ ID NO 24), and the non-alpha-methylated analogue of Formula X, Formula XIII (SEQ ID NO. 25).

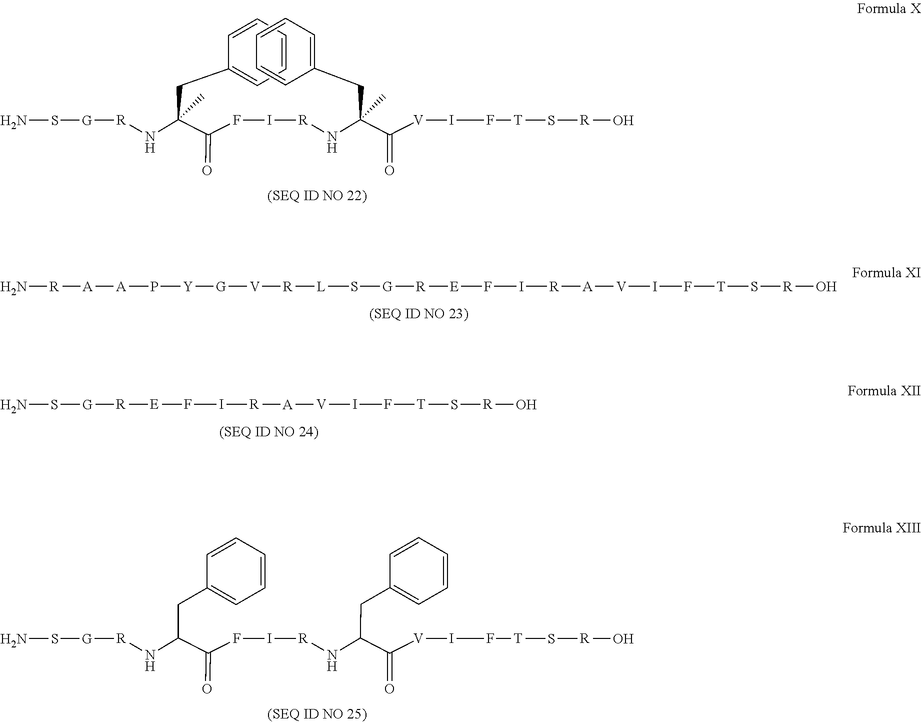

11.1 Binding and cAMP Activity

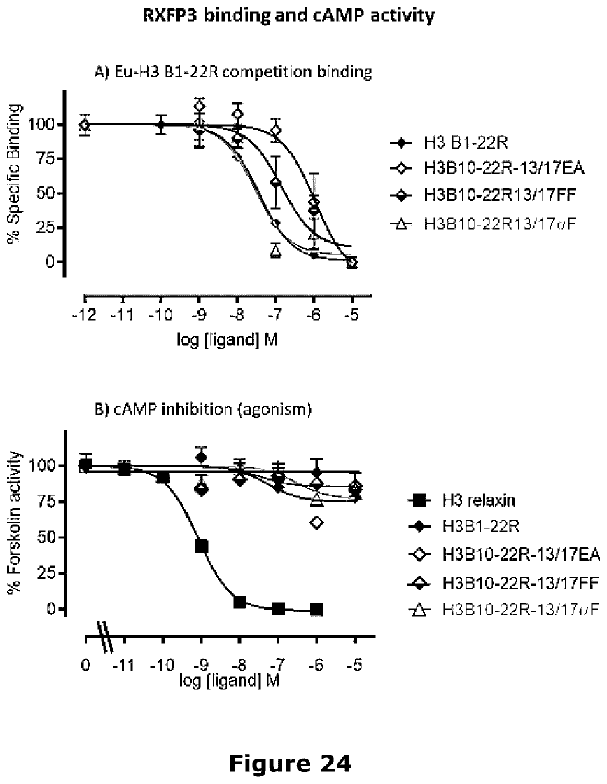

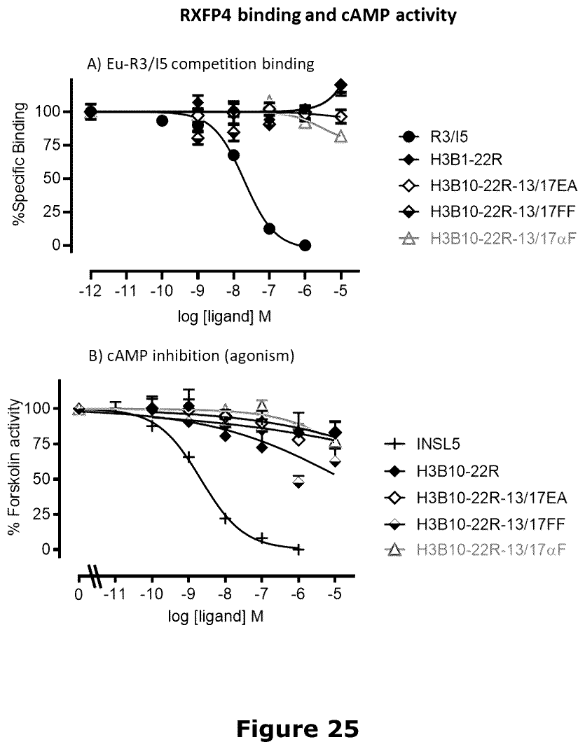

Competition binding and cAMP activity assay were conducted by a procedure analogous to that described above for Example 3. The results are shown in FIG. 24 (RXFP3) and FIG. 25 (RXFP4). Binding affinity (pKi) and cAMP potency (pEC50) data is presented in Table 6.

TABLE 6
Binding (pKi) and cAMP activity (pEC50) of RXFP3 antagonist analogues

| | RXFP3 | RXFP3 | RXFP4 | RXFP4 |
|---|---|---|---|---|
| | Eu-H3B1-22R | cAMP pEC50 | Eu-R3/I5 pKi | cAMP pEC50 |
| Analogues | pKi (n) | (n) | (n) | (n) |
| H3B1-22R | 7.64 ± 0.09 (4) | No activity (3) | No binding (3) | No activity (3) |
| H3B10- | 6.08 ± 0.10 (3)** | No activity (3) | No binding (3) | No activity (3) |
| 22R13/17EA | | | | |
| H3B10- | 6.67 ± 0.54 (3)* | No activity (4) | No binding (3) | No activity (3) |
| 22R13/17FF | | | | |
| H3B10- | 7.60 ± 0.04 (3) | No activity (3) | No binding (3) | <5 (4) |
| 22R13/17αF | | | | |

*p < 0.05,
**p < 0.01 vs H3B1-22R

The Eu-H3B1-22R competition binding assay in CHO-K1-RXFP3 cells suggests that incorporation of α-methyl phenylalanine in i, i+4 manner at positions 13 and 17 results in similar binding to that of H3B1-22R, whereas control peptides (H3B10-22R-13/17FF and H3B10-22R-13/17EA) exhibited a lower binding affinity (FIG. 24A, Table 6). The ability of the analogues to activate the RXFP3 receptor by measuring their ability to inhibit forskolin-stimulated cAMP production in CHO-K1-RXFP3 cells. As expected, none of the analogues was able to activate RXFP3 (FIG. 24B, Table 6), suggesting that the target peptide might be an RXFP3 antagonist. Eu-R3/15 competition binding assay in CHO-K1-RXFP4 cells data suggest that none of the analogues bind to the RXFP4 receptor (FIG. 25A, Table 6). Subsequently, these analogues were tested for their ability to activate the RXFP4 receptor by measuring their ability to inhibit forskolin-stimulated cAMP production in CHO-K1-RXFP4 cells. As expected, none of the analogues exhibited significant activity at the RXFP4 receptor (FIG. 25B, Table 6). Accordingly, it was shown that these antagonist analogues are selective for the RXFP3 receptor.

### 11.2 Antagonism Assay

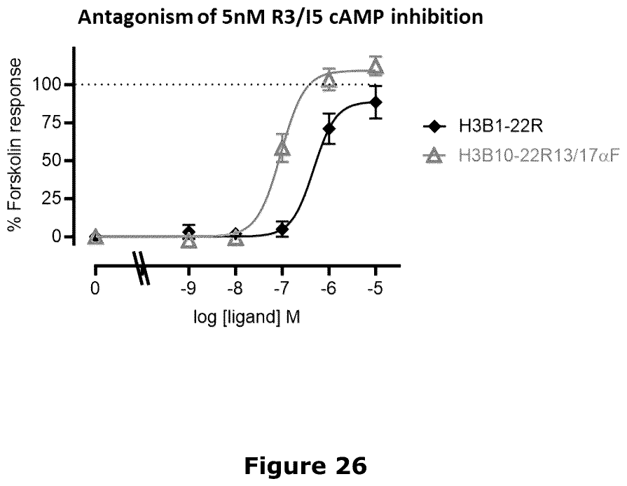

The best performing antagonist H3B10-22R-13/17αF, was tested for its ability to antagonize the function of R3/I5, an RXFP3 agonist. H3B10-22R-13/17αF analogue acts as an RXFP3 antagonist as evidenced by its ability to block the function of 5 nM R3/I5 that is inhibition of the forskolin-induced cAMP activity (FIG. 26, Table 7). Importantly, H3B10-22R-13/17αF demonstrated higher potency and efficacy than H3B1-22R, possibly due to its higher stability in the 6-hour cAMP activity assay.

TABLE 7
Antagonism potency (pIC50) and efficacy
of RXFP3 antagonist analogues

| | RXFP3 Antagonist | RXFP3 Antagonist |
|---|---|---|
| Analogues | potency pIC50 (n) | efficacy (%) (n) |
| H3B1-22R | 6.25 ± 0.10 (3) | 90.9 ± 12.8 (3) |
| H3B10-22R13/17αF | 6.98 ± 0.16 (4)** | 110.6 ± 6.7 (4) |

**p < 0.01 vs H3 B1-22R

### 11.3 Effect of Antagonist on Agonist Induced Chow Consumption

In vivo studies were performed to see the effect of the RXFP3 antagonist, on RXFP3 agonist-induced food intake in adult rats. Normality of data was achieved in the aCSF and 1 nmol RXFP3 agonist groups. Thus, an unpaired t-test was used to compare the means, while for the remainder, median comparisons were made using Mann-Whitney tests. Consistent with previous observations (see above Example 5, and Bathgate et al. Noncovalent Peptide Stapling Using Alpha-Methyl-1-Phenylalanine for α-Helical Peptidomimetics. J. Am. Chem. Soc., 2023, 145, 37, 20242-20247), icv infusion of 1 nmol H3B10-27(13/17αF) (RXFP3 agonist, n=10) significantly increased chow consumption in satiated rats relative to aCSF vehicle controls (n=11; p=0.001, t₁₉=3.75). Infusions of H3B10-22R(13/17αF) (putative RXFP3 antagonist) slightly induced a significant reduction in the chow consumption at a concentration of 2 nmol (n=14, p=0.048, MW U 41). However, no significant reduction was obtained at a 4 nmol dosage (n=14, p=0.10, M-W U 47) relative to the effect of a 1 nmol injection of the agonist. A more significant effect could be observed when comparing the infusion of 8 nmol of H3B10-22R(13/17αF) before 1 nmol agonist (n=12; p=0-005, MW U 22) with the infusion of the agonist alone (n=11). Significant differences were also observed between the groups that received an injection of the antagonist alone (n=12) and the group that received an infusion of the agonist alone (p=0.0003, MW U 12). No significant differences were observed between the aCSF group and any of the groups that received infusions of RXFP3 antagonist. Our studies confirm that the RXFP3 agonist, H3B10-27(13/17αF), can significantly increase food intake, as described, and additionally, that these RXFP3-related increases can be prevented by the prior infusion of the RXFP3 antagonist, H3B10-22R(13/17αF), in a dose-dependent manner. While 2 and 4 nmol antagonist were unable to significantly reduce chow consumption, a higher dose of 8 nmol significantly reduced agonist-induced chow intake. No significant differences were observed between the infusion of aCSF vehicle and any of the combinations in which the antagonist, H3B10-22R(13/17αF), was present.

### CONCLUSION

The present inventors have found a novel stapling strategy that utilizes the helix-inducing properties of both the α-methyl and phenyl moieties in αF. This approach has been successfully applied, by employing a pair of αF, to engineer an π-helical B-chain mimetic of H3 relaxin which, unlike the HC-stapled mimetic, is a single species, water soluble and high-yielding. The novel analogue, H3B10-27(13/17αF), is remarkably stable in serum and mimics full biological function of RXFP3 agonists (R3/I5, A2 and Peptide 5) in both cell-based and animal (ex vivo and in vivo) studies. It is a much simpler scaffold, compared with H3 relaxin, R3/I5 and Peptide 5, to improve its drug properties (e.g., in vivo half-life, CNS delivery). It is also an important tool to elucidate the physiological roles of relaxin-3/RXFP3 neurocircuits. Furthermore, the novel analog H3B10-22R-13/17αF antagonist, is a much simpler scaffold than H3B1-22R, and can be produced in high yields. It was demonstrated to antangonise the agonist function of R3/15, and inhibit RXFP3-related increase in food consumption in an animal in vivo model, suggesting its potential for use in treating e.g. eating related disorders, weight gain related disorders, and addictions.

The stapling approach has also been successfully applied to INSL5 to prepare an π-helical B-chain mimetic, which can be prepared in very high yield at low cost, and exhibits biological activity and high potency. The single chain peptide also provides an easier scaffold to modify for improvement of pharmacokinetics than the two-chain agonist.

The non-covalent stapling method of the present disclosure, unlike covalent stapling methods (e.g., HC-, lactam, thioether etc), is not likely to disrupt the network of stabilizing intramolecular interactions present in the bound state of peptides, and therefore may have general utility in stabilizing wide ranges of biologically important peptide targets.

1. A peptide comprising two α,α-disubstituted amino acid residues, wherein:
the peptide comprises an α-helix; and
the α,α-disubstituted amino acids each comprise as their α-substituents both of:
a) an alkyl, cycloalkyl or alkenyl group; and;
b) an aromatic group capable of π-π stacking with the aromatic group of the other α,α-disubstituted amino acid residue,
and wherein the two α,α-disubstituted amino acid residues are located at residue positions such that, in the α-helix, their aromatic groups may engage in π-π stacking with one another.

2. The peptide according to claim 1, wherein the two α,α-disubstituted amino acid residues are located at residue positions i and i+x, wherein x is 3, 4, 6, 7, 8, 9, 10, 11 or 12.

3. The peptide according to claim 2, wherein the two α,α-disubstituted amino acid residues are located at residue positions i and i+x, wherein x is 3, 4, 6, 7, or 8.

4. The peptide according to claim 3, wherein the two α,α-disubstituted amino acid residues are located at residue positions i and i+x, wherein x is 3 or 4.

5. The peptide according to any one of claims 1 to 4, wherein the peptide is a single-chain peptide.

6. The peptide according to any one of claims 1 to 5, wherein the alkyl, cycloalkyl or alkenyl group is a C₁-C₈ alkyl, cycloalkyl or alkenyl group, a C₁-C₆ alkyl, cycloalkyl or alkenyl group or a C₁-C₄ alkyl, cycloalkyl or alkenyl group.

7. The peptide according to any one of claims 1 to 6, wherein the alkyl group is methyl.

8. The peptide according to any one of claims 1 to 7, wherein the aromatic group is a benzyl group.

9. The peptide according to any one of claims 1 to 8, wherein at least one of the α,α-disubstituted amino acid residues is methyl-L-phenylalanine.

10. The peptide according to claim 9, wherein two of the α,α-disubstituted amino acid residues are methyl-L-phenylalanine.

11. The peptide according to any one of claims 1 to 10, wherein the peptide is selective for a G protein-coupled receptor, preferably wherein the G protein-coupled receptor is an RXFP receptor.

12. The peptide according to any one of claims 1 to 11, wherein the peptide is selective for the RXFP3 receptor or the RXFP4 receptor.

13. The peptide according to any one of claims 1 to 12, wherein the peptide is an analogue of the B chain of an insulin-like peptide or a fragment thereof.

14. The peptide according to any one of claims 1 to 13, wherein the peptide is an analogue of the B chain of insulin-like peptide 7 or a fragment thereof.

15. The peptide according to any one of claims 1 to 14, wherein the peptide is an agonist of the RXFP3 receptor.

16. The peptide according to claim 15, which is a peptide according to SEQ ID NO. 4, or a variant thereof comprising at least about 80% sequence identity to SEQ ID NO. 4.

17. The peptide according to any one of claims 1 to 14, wherein the peptide is an antagonist of the RXFP3 receptor.

18. The peptide according to claim 17, which is a peptide according to SEQ ID NO. 22, or a variant thereof comprising at least about 80% sequence identity to SEQ ID NO. 22.

19. The peptide according to any one of claims 1 to 13, wherein the peptide is an analogue of the B chain of insulin-like peptide 5 or a fragment thereof.

20. The peptide according to any one of claims 1 to 13 or claim 19, wherein the peptide is an agonist of the RXFP4 receptor.

21. The peptide according to claim 19 or claim 20, wherein residues 20 and 21 are each substituted with Gly.

22. The peptide according to claim 21, which is a peptide according to SEQ ID NO. 16, or a variant thereof comprising at least about 80% sequence identity to SEQ ID NO 16.

23. The peptide according to claim 20 or claim 21, wherein residue 19 is substituted with glutamic acid.

24. The peptide according to claim 23, which is a peptide according to SEQ ID NO. 26, or a variant thereof comprising at least about 80% sequence identity to SEQ ID NO. 26.

25. A pharmaceutical composition comprising a peptide according to any one of claims 1 to 24 and a pharmaceutically acceptable carrier, excipient, diluent, vehicle, and/or adjuvant.

26. A method of treating or preventing a condition associated with RXFP activity, preferably RXFP3 or RXFP4 activity, comprising administering to a subject in need thereof an effective amount of a peptide according to any one of claims 12 to 24 or the pharmaceutical composition of claim 25.

27. The method according to claim 26, wherein the condition is associated with RXFP3 activity, preferably wherein the peptide is a peptide according to claims 14 to 18.

28. The method according to claim 27, wherein the condition is selected from eating disorder, weight loss, weight gain, or obesity.

29. The method according to claim 27, wherein the condition is pain.

30. The method according to claim 27, wherein the condition is a neuropsychiatric disorder.

31. The method according to claim 30, wherein the neuropsychiatric disorder is selected from anxiety and depression.

32. The method according to claim 26, wherein the condition is associated with RXFP4 activity, preferably wherein the peptide is a peptide according to any one of claims 19 to 24.

33. The method according to claim 32, wherein the condition is a colon motility disorder.

34. Use of a peptide according to any one of claims 12 to 24 in the manufacture of a medicament for the treatment or prevention of a condition associated with RXFP activity, preferably RXFP3 or RXFP4 activity.

35. The use according to claim 34, wherein the condition is associated with RXFP3 activity, preferably wherein the peptide is a peptide according to any one of claims 14 to 18.

36. The use according to claim 35, wherein the condition is selected from an eating disorder, weight loss, weight gain, or obesity.

37. The use according to claim 35, wherein the condition is pain.

38. The use according to claim 35, wherein the condition is a neuropsychiatric disorder.

39. The use according to claim 38, wherein the neuropsychiatric disorder is selected from depression and anxiety.

40. The use according to claim 34, wherein the condition is associated with RXFP4 activity, preferably wherein the peptide is a peptide according to any one of claims 19 to 24.

41. The use according to claim 40, wherein the condition is a colon motility disorder.


SEQUENCE LISTING (26 sequences)

SEQ ID NO: 1 - protein, 24 aa, synthetic construct
  DISULFID 10..15: note = Disulfide bond between Cys 10 and Cys 15
  CROSSLNK 11: note = Disulfide bond between Cys 11 of SEQ ID No:1 and Cys 10 of SEQ ID No:2
  CROSSLNK 24: note = Disulfide bond between Cys 24 of SEQ ID No:1 and Cys 22 of SEQ ID No:2
DVLAGLSSSCCKWGCSKSEISSLC

SEQ ID NO: 2 - protein, 27 aa, synthetic construct
  CROSSLNK 10: note = Disulfide bond between Cys 10 of SEQ ID No:1 and Cys 11 of SEQ ID No:2
  CROSSLNK 22: note = Disulfide bond between Cys 22 of SEQ ID No:1 and Cys 24 of SEQ ID No:2
RAAPYGVRLCGREFIRAVIFTCGGSRW

SEQ ID NO: 3 - protein, 18 aa, synthetic construct
  SITE 4: note = X is alpha-methyl-L-norleucine
  CROSSLNK 4..8: note = carbon-carbon double bond between the side chains of residues 4 and 8
  SITE 8: note = X is alpha-methyl-L-phenylalanine
SGRXFIRXVIFTSGGSRW

SEQ ID NO: 4 - protein, 18 aa, synthetic construct
  SITE 4: note = X is alpha-methyl-L-phenylalanine
  SITE 8: note = X is alpha-methyl-L-phenylalanine
SGRXFIRXVIFTSGGSRW

SEQ ID NO: 5 - protein, 18 aa, synthetic construct
SGRFFIRFVIFTSGGSRW

SEQ ID NO: 6 - protein, 18 aa, synthetic construct
SGREFIRAVIFTSGGSRW

SEQ ID NO: 7 - protein, 21 aa, synthetic construct
  DISULFID 7..12: note = Disulfide bond between Cys 7 and Cys 12
  CROSSLNK 8: note = Disulfide bond between Cys 8 of SEQ ID No:7 and Cys 7 of SEQ ID No:8
  CROSSLNK 21: note = Disulfide bond between Cys 21 of SEQ ID No:7 and Cys 19 of SEQ ID No:8
ZDLQTLCCTDGCSMTDLSALC

SEQ ID NO: 8 - protein, 24 aa, synthetic construct
  CROSSLNK 7: note = Disulfide bond between Cys 7 of SEQ ID No:8 and Cys 8 of SEQ ID No:7
  CROSSLNK 19: note = Disulfide bond between Cys 19 of SEQ ID No:8 and Cys 21 of SEQ ID No:7
KESVRLCGLEYIRTVIYICASSRW

SEQ ID NO: 9 - protein, 14 aa, synthetic construct
  CROSSLNK 14: note = Disulfide bond between Cys 14 and Cys 19 of SEQ ID No:10
  CROSSLNK 1: note = Disulfide bond between Cys 1 and Cys 7 of SEQ ID No:10
CTDGASMKDLSALC

SEQ ID NO: 10 - protein, 24 aa, synthetic construct
  CROSSLNK 7: note = Disulfide bond between Cys 7 and Cys 1 of SEQ ID No:9
  CROSSLNK 19: note = Disulfide bond between Cys 19 and Cys 14 of SEQ ID No:9
KESVRLCGLEYIRTVIYICASSRW

SEQ ID NO: 11 - protein, 14 aa, synthetic construct
  CROSSLNK 1: note = Disulfide bond between Cys 1 and Cys 1 of SEQ ID No:12
  CROSSLNK 14: note = Disulfide bond between Cys 14 and Cys 13 of SEQ ID No:12
CTDGASMKDLSALC

SEQ ID NO: 12 - protein, 18 aa, synthetic construct
  CROSSLNK 1: note = Disulfide bond between Cys 1 and Cys 1 of SEQ ID No:11
  CROSSLNK 14: note = Disulfide bond between Cys 13 and Cys 14 of SEQ ID No:11
CGLEYIRTVIYICASSRW

SEQ ID NO: 13 - protein, 14 aa, synthetic construct
  CROSSLNK 1: note = Disulfide bond between Cys 1 and Cys 1 of SEQ ID No:14
  CROSSLNK 14: note = Disulfide bond between Cys 14 and Cys 13 of SEQ ID No:14
CTDGASMKDLSALC

SEQ ID NO: 14 - protein, 18 aa, synthetic construct
  CROSSLNK 1: note = Disulfide bond between Cys 1 and Cys 1 of SEQ ID No:13
  CROSSLNK 13: note = Disulfide bond between Cys 13 and Cys 14 of SEQ ID No:13
CGLEYIRTVIYICGGSRW

SEQ ID NO: 15 - protein, 18 aa, synthetic construct
SGLEYIRTVIYISGGSRW

SEQ ID NO: 16 - protein, 18 aa, synthetic construct
  SITE 4: note = X is alpha-methyl-L-phenylalanine
  SITE 8: note = X is alpha-methyl-L-phenylalanine
SGLXYIRXVIYISGGSRW

SEQ ID NO: 17 - protein, 18 aa, synthetic construct
  SITE 1: note = acetylation
  SITE 4: note = X is alpha-methyl-L-phenylalanine
  SITE 8: note = X is alpha-methyl-L-phenylalanine
SGLXYIRXVIYISGGSRW

SEQ ID NO: 18 - protein, 18 aa, synthetic construct
  SITE 1: note = acetylation
  SITE 4: note = X is alpha-methyl-L-norleucine
  SITE 8: note = X is alpha-methyl-L-norleucine
  CROSSLNK 4..8: note = carbon-carbon double bond between the side chains of residues 4 and 8
SGLXYIRXVIYISGASRW

SEQ ID NO: 19 - protein, 19 aa, synthetic construct
  CROSSLNK 4..8: note = lactam bridge between the side chains of Lys 4 and Asp 8
SGLKYIRDVIYISGASRWH

SEQ ID NO: 20 - protein, 18 aa, synthetic construct
  SITE 4: note = X is alpha-methyl-L-cysteine
  SITE 8: note = X is alpha-methyl-L-cysteine
  CROSSLNK 4..8: note = Thioacetal bond between the side chain of residues 4 and 8
  SITE 1: note = acetylation
SGLXYIRXVIYISGGSRW

SEQ ID NO: 21 - protein, 18 aa, synthetic construct
  SITE 1: note = acetylation
  SITE 4: note = X is alpha-methyl-L-cysteine
  SITE 8: note = X is alpha-methyl-L-cysteine
  DISULFID 4..8: note = Disulfide bond between residues 4 and 8
SGLXYIRXVIYISGGSRW

SEQ ID NO: 22 - protein, 14 aa, synthetic construct
  SITE 4: note = X is alpha-methyl-L-phenylalanine
  SITE 8: note = X is alpha-methyl-L-phenylalanine
SGRXFIRXVIFTSR

SEQ ID NO: 23 - protein, 22 aa, synthetic construct
RAAPYGVRLSGREFIRAVIFTS

SEQ ID NO: 24 - protein, 14 aa, synthetic construct
SGREFIRAVIFTSR

SEQ ID NO: 25 - protein, 14 aa, synthetic construct
SGRFFIRFVIFTSR

SEQ ID NO: 26 - protein, 18 aa, synthetic construct
  SITE 4: note = alpha methyl L phenylalanine
  SITE 8: note = alpha methyl L phenylalanine
SGLXYIRXVIYIEGGSRW